(19) United States
(12) Patent Application Publication
(10) Pub. No.: US 2022/0184007 A1
(43) Pub. Date: Jun. 16, 2022

(54) FORMULATIONS

(71) Applicant: Synchroneuron Inc., Lexington, MA (US)

(72) Inventors: Barry S. Fogel, Lexington, MA (US); William D. Kerns, DVM, MS, DACVP, Duxbury, MA (US); Kei-Lai Fong, Duxbury, MA (US)

(73) Assignee: Synchroneuron Inc., Lexington, MA (US)

(21) Appl. No.: 17/599,673

(22) PCT Filed: Apr. 2, 2020
(86) PCT No.: PCT/US20/26369

§ 371 Date: Sep. 29, 2021

Related U.S. Application Data
(60) Provisional application No. 62/828,507, filed on Apr. 3, 2019.

(51) Int. Cl. A61K 31/16; A61K 9/20; A61K 9/00

(52) U.S. Cl. CPC A61K 31/16; A61K 9/2054; A61K 9/2027; A61K 9/2013; A61K 9/0053; A61K 9/2031; A61K 9/2009; A61K 9/2095

(57) ABSTRACT

Provided herein are high-dose formulations of acamprosate in a pharmaceutically acceptable salt form, as well as methods of preparing the same, and methods of using the same.

### RELATED APPLICATIONS

This application claims priority to U.S. Provisional Application No. 62/828,507, filed Apr. 3, 2019, the entire contents of which are hereby incorporated by reference.

### BACKGROUND

The formulation of drug products, and particularly of drug products containing a large dose of active pharmaceutical ingredient (“API”), is a challenging area of drug research (Sun, C. C. et al. Development of a High Drug Load Tablet Formulation Based on Assessment of Powder Manufacturability: Moving Towards Quality by Design, Journal of Pharmaceutical Sciences, Vol. 98, 239-247 (2009)). Particularly for oral formulations, the necessity of a high dose of API limits the amounts of excipients that can be included (e.g., to ensure appropriate product characteristics and uniformity), because tablets (or capsules) larger than about 13 mm in maximum dimension can be difficult or impossible for patients to swallow (Fields, J. et al. Pill Properties that Cause Dysphagia and Treatment Failure, Current Therapeutic Research, Vol. 77, 79-82 (2015)).

### SUMMARY

The present disclosure provides description of surprisingly useful pharmaceutical formulations, that achieve particular desirable results even when used to formulate high dose oral compositions, and particularly high dose tablet compositions (e.g., tablets or mini-tablets).

The present disclosure builds upon surprising findings, demonstrated in WO 2013/082573 and WO 2014/197744 (the “Prior Fogel Filings”), that certain polymers have unique properties that render them particularly useful and/or effective in the preparation of certain pharmaceutical compositions. In particular, the Prior Fogel Filings demonstrate that certain cross-linked high molecular weight polymers of acrylic acid, known as carbomers, and particularly polymers that are homopolymer type B carbomers or homopolymer type A carbomers, can meet stringent requirements for drug product properties (related to e.g., hardness, firmness, friability, and/or resistance to erosion) and also achieve release profiles that permit new dosing formats and/or regimens for certain pharmaceutical agents, specifically including certain pharmaceutical agents such as acamprosate (e.g., acamprosate calcium). In some embodiments, such new formats and/or regimens permit treatment of different diseases, disorders and conditions and/or permit treatment of particular patient populations. In some embodiments, such new formats and/or regimens show superior tolerability, side effect profiles, and/or increased convenience for patients versus immediate release formulations of the same pharmaceutical agents.

As mentioned in the Prior Fogel Filings, acamprosate calcium, because of its actions on glutamate and GABA transmission, can correct imbalances of excitatory (glutamate-mediated) and inhibitory (GABA-mediated) neurotransmission. Such imbalances are currently thought to play a role in causing or influencing the severity of diverse neurological and psychiatric conditions involving recurrent, involuntary, stereotypical, and unwanted movements, perceptions, thoughts and behaviors, including tardive dyskinesia (TD), levodopa-induced dyskinesia (LID), Tourette Syndrome (TS), obsessive-compulsive disorder (OCD), posttraumatic stress disorder (PTSD), tinnitus, autism, and addictions to alcohol, nicotine, and cocaine. Several U.S. patents (e.g., U.S. Pat. Nos. 6,057,373, 6,294,583, 6,391,922, 6,689,816, and 7,498,361; each of which is incorporated herein by reference in its entirety) describe the use of acamprosate to treat neuropsychiatric disorders, including tardive dyskinesia and other movement disorders induced by chronic exposure of patients to neuroleptic (antipsychotic) drugs, Tourette syndrome, and mental disorders such as posttraumatic stress disorder (PTSD) and obsessive-compulsive disorder (OCD).

In addition to the above-mentioned neurological and psychiatric conditions, it is postulated that an acamprosate agent such as acamprosate calcium may be useful in the treatment of Psychogenic Nonepileptic Seizures (PNES). PNES were first described in the medical literature of the 19^{th} century as seizure-like attacks that were not related to an identified central nervous system lesion. However, despite the condition being known for many decades, very little is known about what causes such seizures. Currently, PNES is classified as a conversion disorder in the Diagnostic and Statistical Manual of Mental Disorders (DSM-5). The condition affects about 100,000 patients in the US. Few studies have been undertaken to evaluate the effectiveness of psychotropic medications in the treatment of PNES. One study by La France et al. evaluated the effectiveness of flexible-dose sertraline over 12 weeks in reducing event frequency and improving psychiatric co-morbidities and psychosocial domains (LaFrance W, Keitner G, Papandonatos G, Blum A, Machan J, Ryan C, Miller I. Pilot pharmacologic randomized controlled trial for psychogenic nonepileptic seizures. Neurology. 2010; 75:1166-1173). However, the final analysis was statistically underpowered (33 subjects) and showed no difference between groups on biweekly event frequency change. An open-label study of flexible-dose venlafaxine resulted in significant event reduction, however, this was an uncontrolled study (Pintor L, Bailles E, Matrai S, Carreno M, Donaire A, Boget T, Setoain X, Rumia J, Bargalló N. Efficiency of venlafaxine in patients with psychogenic nonepileptic seizures and anxiety and/or depressive disorders. Journal of Neuropsychiatry and Clinical Neurosciences, 2010; 22:401-408). Without wishing to be bound by any theory, the present disclosure provides an insight that PNES may be characterized by recurrent, involuntary, stereotypical and unwanted movements and behavior, and thus, an agent (e.g., an acamprosate agent) that corrects imbalances of glutamate-mediated excitation ad GABA-mediated inhibition may be useful for the treatment of PNES. Accordingly, in some embodiments, the present disclosure includes the recognition that acamprosate is useful for the treatment of PNES.

Among other things, the Prior Fogel Filings specifically demonstrated that homopolymer type B carbomers and homopolymer type A carbomers can be particularly useful when preparing high dose formulations, and particularly when preparing tablet formulations containing a high dose of active agent (e.g., tablet formulations in which a significant percentage of the weight of the tablet is comprised of active agent). In particular, the development of a robust tablet formulation for a high dose active pharmaceutical ingredient (API) is challenging as such formulations usually lack the properties to be formed into tablets. The necessity of the formulation consisting of such a high percentage of drug by weight leaves little room for the addition of necessary agents (e.g., excipients) for the production of tablets with suitable physical properties. Adding the requirement for a sustained release formulation exacerbates the problem, as additional release characteristics (e.g., specified release profiles) must be attained. The Prior Fogel Filings demonstrated that acamprosate calcium could be formulated as an extended release high drug dose tablet with the addition of low weight percentages of homopolymer type B carbomers or homopolymer type A carbomers. The carbomer class of polymer has been found to exhibit surprising results in this regard. Without being bound by any particular theory, there are unique and surprising positive interactions between acamprosate and the carbomers that result in the superior physical characteristics and dissolution profiles seen. The combination of acamprosate with a multitude of other polymers cannot duplicate these positive results.

A common problem with the oral administration of high doses of active agent is the phenomenon of dose dumping. Dose dumping is a phenomenon in which various factors can cause premature and exaggerated release of the active agent. This large increase in concentration of the active agent in the body can produce adverse effects and in some cases toxicity. Dose dumping is most commonly seen in oral doses of active agent which are absorbed in the gastrointestinal tract, where in some cases, sudden release of the active agent occurs within seconds in a concentrated area. High local concentrations of the active agent can cause irritation (e.g., irritation of the stomach and/or large intestine) and adverse side effects, such as nausea, vomiting, abdominal pain and/or diarrhea. In some cases, increases in the level of active agent may saturate an uptake transporter responsible for the absorption of the active agent. This can result in larger quantities of unabsorbed active agent passing to the lower gastrointestinal tract which may lead to adverse side effects, such as diarrhea, and/or can result in reduced overall bioavailability. Such side effects may preclude administration of the active agent at a high enough dose to be efficacious for the treatment of disease (e.g., when a particular threshold concentration of active agent in the blood is necessary to achieve sufficient penetration of the blood-brain barrier, and/or when such threshold concentration must be maintained over a particular period of time for a desired effect to be achieved in the brain). Particular pharmaceutical formulations (e.g., particular excipients and coatings) may affect the extent of dose dumping. For example, Campral® is a delayed-release formulation of acamprosate calcium, which may suffer from the drawbacks of dose dumping in patients. The present disclosure allows for the extended release of active agent which may allow for higher doses and avoid dose dumping and its associated side effects. This may allow for the treatment of conditions which were previously untreatable due to complications involving dose dumping at the higher amounts of active agent required for treatment.

A further issue that can negatively impact the oral administration of high doses of active agent is a negative food effect. That is, a decrease in bioavailability, upon the co-administration of the active agent together with food. This may be due to intestinal dilution which leads to a lower concentration gradient across the intestinal wall as well as the active agent binding to food components which reduces the fraction of active agent available for absorption. This effect can be especially pronounced with active agents that are high solubility/low permeability compounds (BCS class III). In some cases, a food effect is observed when the active agent (e.g., acamprosate) is absorbed via amino acid carrier transport processes and therefore must compete with the protein from food for transport. The present disclosure allows for the extended release of active agent which may allow for higher doses and avoid a negative food effect. This may allow for the treatment of conditions which were previously untreatable due to complications involving a negative food effect.

Large dose drugs also pose the challenge of tablet size. The additional agents by necessity need to remain at a low weight percentage as otherwise the tablet size will become too large. If the tablet size becomes large enough it may be difficult to swallow, thereby diminishing its use as an orally administered therapy. In certain patient populations (including but not limited to children and elderly patients) swallowing large pills poses a problem, particularly with respect to, e.g., the maximum dose that can be administered and/or patient compliance. In some embodiments, the present disclosure provides formulations in which active agent constitutes a significant percentage (e.g., about 30 wt %, about 40 wt %, about 50 wt %, about 60 wt %, about 70 wt %, or about 80 wt %) of the formulation (e.g., by weight). For example, in some embodiments, provided formulations minimize addition of agents (e.g., excipients) other than the active agent. As those skilled in the art are aware, such formulations can be difficult to develop, as agents (e.g., excipients) other than the active agent are commonly required to achieve desirable or necessary structural, material, and/or functional (e.g., delivery rate and/or other characteristics) attributes of a formulation.

Those skilled in the art are aware that a tablet composition with a diameter of <13 mm (e.g., <8 mm) is typically considered to be easily swallowed; tablets above this size may present particular challenges related to their ability to be readily swallowed. The FDA has established guidelines indicating that tablets having a largest dimension no greater than 22 mm are considered to be acceptable by the FDA (Size, Shape, and Other Physical Attributes of Generic Tablets and Capsules—Guidance for Industry, Food and Drug Administration, June 2015). The Prior Fogel Filings describe certain high-dose acamprosate formulation(s) that surprisingly achieve good swallowability even with a longest dimension above 13 mm, or even above 15 mm or above 18 mm (e.g., 20 mm or more). In particular, the Prior Fogel Filings describe oval-shaped tablets with a longest dimension materially greater than 13 mm that are characterized by good swallowability. The present disclosure surprisingly demonstrates that other acamprosate formulations can also achieve good swallowability notwithstanding a longest dimension above 13 mm, or even above 15 mm or above 18 mm (e.g., within a range of 20-22 mm, or specifically about 20 mm).

The present disclosure surprisingly demonstrates that suitable formulations for the preparation of high dose tablets are not limited solely to the particular carbomer homopolymers exemplified in the Prior Fogel Filings. Rather, the present disclosure demonstrates that, although many oft-utilized pharmaceutical polymer systems cannot be successfully employed to produce high dose formulations suitable for the preparation of tablet compositions (e.g., tablets and/or mini-tablets), at least certain type A, B, and C carbomer homopolymers can.

Among other things, the present disclosure defines certain specifications for particularly useful tablet compositions of relevant pharmaceutical agents.

For example, the present disclosure demonstrates that formulations meeting the performance and material requirements described herein can be prepared using any class of carbomer (i.e., carbomer homopolymers Type A, Type B, or Type C), so long as they are present within a particular range (e.g., within about 20 mg to about 130 mg in a tablet with total (uncoated) mass within the range of about 950 mg to about 1200 mg). Without wishing to be bound by any particular theory, the present disclosure discloses that weight percent of the carbomer in the tablet composition may be a relevant feature; in many embodiments, provided formulations include between about 1.8% and about 11.6% of carbomer by weight of tablet composition, absent any coating. In some embodiments, provided formulations include between about 5.4% and about 7.1% of carbomer homopolymer type A by weight of tablet composition, absent any coating. In some embodiments, provided formulations include between about 3.2% and about 11.6% of carbomer homopolymer type B by weight of tablet composition, absent any coating. In some embodiments, provided formulations include between about 1.8% and about 11.2% of carbomer homopolymer type C by weight of tablet composition, absent any coating.

Alternatively or additionally, without wishing to be bound by any particular theory, the present disclosure proposes that the relative amount of active ingredient to carbomer may be a relevant feature; in many embodiments, provided formulations include active:carbomer ratio within a range of about 5:1 to about 14:1 for carbomer homopolymer type A. In some embodiments, provided formulations include active:carbomer ratio within a range of about 3:1 to about 23:1 for carbomer homopolymer type B. In some embodiments, provided formulations include active:carbomer ratio within a range of about 3:1 to about 40:1 for carbomer homopolymer type C.

In addition to demonstrating the surprising utility of all classes of carbomers (i.e., not solely carbomer homopolymer type B) the present disclosure demonstrates the failure of certain other compositions, including certain formulations comprising a polymer matrix of polyethylene-oxide-containing compositions, notwithstanding recommendations in the art that polyethylene oxide polymers (PEO) such as POLYOX® are particularly useful in formulations of active agents including acamprosate (see, for example, Berner US 2012/0077878).

Moreover, the present disclosure demonstrates that other known polymers (e.g., carboxypropylmethylcellulose) do not produce useful formulations that meet all requirements as set out herein. That is, the present disclosure demonstrates that, even when a composition meets indicated dissolution criteria, it may not be useful for treatment as described herein, among other things, due to failure of material properties and/or other characteristics. In some instances, the failure may be due to lack of suitable hardness, swelling or other properties. In some instances, the tablets may have an eroded surface or a weaker gel coat.

Without wishing to be bound by any particular theory, the present disclosure discloses that carbomers' unique usefulness as a class may be attributable, for example, to unique and surprising positive interactions between the active agent (e.g., acamprosate) and the carbomers that dictate the positive physical characteristics and dissolution profiles described herein, which are not duplicated with other polymers.

Carbomers are a negatively charged polymers that are efficient matrix-forming agents. Carbomers have a very high molecular weight, due to extensive cross-linking, and the average molecular weight of a carbomer is calculated to be 3-4.5 billion Da. Due to extensive cross-linking, carbomers are not soluble, but only swellable in water. The polymers swell to form a hydrated matrix layer. The resulting hydrogel formed is not comprised of single entangled chains of polymers but rather is comprised of discrete microgels made up of many polymer particles in which the drug is dispersed. The hydrated matrix layer controls water penetration and the diffusion of drug through the hydrated matrix. At higher pH, carbomers form gels which further retard the release of the pharmaceutical agent. The present disclosure documents that certain other negatively charged polymers which may also be soluble at higher pH, (e.g., alginate, carboxymethyl cellulose, and EUDRAGIT® L) do not achieve useful formulations as described herein. Without wishing to be bound by any particular theory, we note that these other polymers do not have the high molecular weight (i.e., within the range of about 3 billion Da to about 4.5 billion Da overall; and about 3 billion Da to about 4.5 billion Da overall for carbomer homopolymers Type A, Type B and Type C). The present disclosure discloses that the precise balance of molecular weight, negative charge, and high-pH gel forming ability found in certain carbomers may be relevant to achievement of the properties and characteristics described herein.

Alternatively or additionally, without wishing to be bound by any particular theory, the present disclosure proposes that formulations of the current disclosure are especially well-suited to acidic active agents.

In some embodiments, an active agent (e.g., an acidic active agent) is provided and/or utilized in accordance with the present disclosure in the form of a pharmaceutically acceptable salt. In some embodiments, an active agent (e.g., acidic active agent) is provided and/or utilized in accordance with the present disclosure in the form of a free acid. In some embodiments, an active agent (e.g., acidic active agent) is provided and/or utilized in accordance with the present disclosure in the form of a pharmaceutically acceptable salt (e.g., calcium salt).

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

### Definitions

Acamprosate Agent: The term “acamprosate agent” as used herein refers to a compound or entity that, when administered to a subject, delivers to that subject an acamprosate active moiety. In some embodiments, an acamprosate agent is or comprises acamprosate. In some embodiments, an acamprosate agent is or comprises a prodrug of acamprosate. In some embodiments, an acamprosate agent is provided and/or utilized in a salt form. In some embodiments, an acamprosate agent is provided and/or utilized as a calcium salt form. In some embodiments, an acamprosate agent is provided and/or utilized as a magnesium salt form, e.g., particularly if a subject has a movement disorder (e.g., tardive dyskinesia or levodopa-induced dyskinesia in patients with Parkinson's disease). In some embodiments, an acamprosate agent is provided and/or utilized as a lithium salt form, e.g., particularly if a subject would benefit from mood stabilization. In some embodiments, an acamprosate agent is provided and/or utilized in a formulation. In some embodiments, an acamprosate agent is provided and/or utilized in a tablet.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Combination therapy: As used herein, the term “combination therapy” refers to those situations in which a subject is simultaneously exposed to two or more therapeutic regimens (e.g., two or more therapeutic agents). In some embodiments, two or more agents or may be administered simultaneously; in some embodiments, such agents may be administered sequentially; in some embodiments, such agents are administered in overlapping dosing regimens.

Comparable: As used herein, the term “comparable” refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form: (or “unit dosage form”), As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: (or “therapeutic regimen”), As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Encapsulated: The term “encapsulated” is used herein to refer to substances that are completely surrounded by another material.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Hyperkinetic movement disorders: As used herein, the term “hyperkinetic movement disorders” refers to disorders such as ataxia, chorea, dystonia, hemifacial spasm, Huntington's disease (HD), myoclonus, restless legs syndrome, tardive dyskinesia or tardive dystonia, tics, Tourette syndrome, tremor, and Wilson's disease.

In vitro: The term “in vitro” as used herein refers to events that occur in an artificial environment, e.g., in a test tube or reaction vessel, in cell culture, etc., rather than within a multi-cellular organism.

In vivo: As used herein refers to events that occur within a multi-cellular organism, such as a human and a non-human animal. In the context of cell-based systems, the term may be used to refer to events that occur within a living cell (as opposed to, for example, in vitro systems).

Isomer: As is known in the art, many chemical entities (in particular many organic molecules and/or many small molecules) can exist in a variety of structural and/or optical isomeric forms. In some embodiments, as will be clear to those skilled in the art from context, depiction of or reference to a particular compound structure herein may represent all structural and/or optical isomers thereof. In some embodiments, as will be clear to those skilled in the art from context, depiction of or reference to a particular compound structure herein is intended to encompass only the depicted or referenced isomeric form. In some embodiments, compositions including a chemical entity that can exist in a variety of isomeric forms include a plurality of such forms; in some embodiments such compositions include only a single form. For example, in some embodiments, compositions including a chemical entity that can exist as a variety of optical isomers (e.g., stereoisomers, diastereomers, etc.) include a racemic population of such optical isomers; in some embodiments such compositions include only a single optical isomer and/or include a plurality of optical isomers that together retain optical activity.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt” or “pharmaceutically acceptable salt form”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: as used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial numerical similarity: As used herein, the term “substantial numerical similarity” refers to two values, for example, two percentages of total drug released from a tablet, having a numerical value that does not differ by more than 30%.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, is meant an amount that produces the desired effect for which it is administered. In some embodiments, the term “therapeutically effective amount” or “therapeutically effective dose” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treatment: As used herein, the term “treatment” (also “treat” or “treating”) refers to any administration of a substance (e.g., an acamprosate agent) that partially or completely alleviates, ameliorates, relieves, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE DRAWING

FIG. 1A depicts the dissolution profile of round 800 mg tablets of Example 1 (Formulation 2) in 0.1N HCl versus oval 800 mg tablets of Example 1 prepared with by (i) extrusion or (ii) milling without a screen (see Example 4).

FIG. 1B depicts the dissolution profile of round 800 mg tablets of Example 1 in pH 4.5 acetate buffer versus oval 800 mg tablets of Example 1 prepared with by (i) extrusion or (ii) milling without a screen (see Example 4).

FIG. 2A depicts the dissolution profile of round 800 mg tablets of Example 1 in 0.1N HCl versus oval 800 mg tablets of Example 1, prepared according to modification (d) of Example 4.

FIG. 2B depicts the dissolution profile of round 800 mg tablets of Example 1 in pH 4.5 acetate buffer versus oval 800 mg tablets of Example 1 prepared according to modification (d) of Example 4.

### DETAILED DESCRIPTION OF CERTAIN EMBODIMENTS

In general, the present disclosure provides new pharmaceutical compositions that comprise one or more active agents in a high dose (e.g., tablet formulations in which a significant percentage of the weight of the tablet is comprised of active agent). The present disclosure also provides technologies for preparing such compositions, and furthermore defines and describes certain performance and physical characteristics of particularly useful compositions.

In some embodiments, teachings of the present disclosure are particularly applicable to tablet compositions, which may be coated or uncoated.

In some embodiments, teachings of the present disclosure are particularly applicable to tablet compositions containing acidic active agents. In some embodiments, the acidic active agent is present as a pharmaceutically acceptable salt form.

In some embodiments, an active agent is administered in a high dose. In some embodiments, an active agent is administered in a dosage form containing a dose of about 400 mg or higher. In some embodiments, an active agent is administered in a dosage form containing a dose of about 800 mg or higher. In some embodiments, such a dosage form is a tablet containing a dose of about 800 mg or higher.

In some embodiments, an active agent is administered in a fed mode. In some embodiments, an active agent is administered is a fasted mode. In some embodiments, an active agent can be administered with or without food with no adverse effect on bioavailability or efficacy. In some embodiments, the release profile is substantially the same in either fed or fasted mode.

In some embodiments, an active agent is administered as multiple dosage forms in a single administration. In some embodiments, an active agent is administered once a day. In some embodiments, an active agent is administered twice a day. In some embodiments, an active agent is administered three times a day. In some embodiments, an active agent is administered multiple times a day.

The teachings of the present disclosure may be particularly useful for compositions that contain high doses of active agent. For example, in some embodiments, provided dosage forms include an active agent in a dose of at least 400 mg. In some embodiments, provided dosage forms include an active agent in a dose of at least 500 mg. In some embodiments, provided dosage forms include an active agent in a dose of at least 600 mg. In some embodiments, provided dosage forms include an active agent in a dose of at least 700 mg. In some embodiments, provided dosage forms include an active agent in a dose of at least 800 mg. In some embodiments, provided dosage forms include an active agent in a dose of at least 900 mg. In some embodiments, provided dosage forms include an active agent in a dose of at least 1000 mg.

In some embodiments, provided compositions include an active agent at a dose that is at least 30% w/w of total weight of the dosage form. In some embodiments, provided compositions include an active agent at a dose that is at least 40% w/w of total weight of the dosage form. In some embodiments, provided compositions include an active agent at a dose that is at least 50% w/w of total weight of the dosage form. In some embodiments, provided compositions include an active agent at a dose that is at least 60% w/w of total weight of the dosage form. In some embodiments, provided compositions include an active agent at a dose that is at least 70% w/w of total weight of the dosage form. In some embodiments, provided compositions include an active agent at a dose that is at least 80% w/w of total weight of the dosage form. In some embodiments, provided compositions include an active agent at a dose that is at least 90% w/w of total weight of the dosage form. In some embodiments, provided compositions include an active agent at a dose that is at least 95% w/w of total weight of the dosage form.

In some embodiments, an active agent is acamprosate. In some embodiments, an active agent may be or comprise an active metabolite thereof. In some embodiments, an active agent is an acamprosate agent. In some embodiments, an acamprosate agent is or comprises a prodrug of acamprosate. In some embodiments, an acamprosate agent is provided and/or utilized in a salt form. In some embodiments, an active agent is acamprosate calcium. In some embodiments, an active agent is acamprosate magnesium. In some embodiments, an active agent is acamprosate lithium. In some embodiments, an active agent is acamprosate sodium. In some embodiments, an acamprosate agent is provided and/or utilized in a formulation. In some embodiments, an acamprosate agent is provided and/or utilized in a tablet composition.

Among other things, the present disclosure demonstrates that carbomers are particularly well suited for the preparation of high dose, extended release tablets. In some embodiments, the provided formulations result in improved tolerability as demonstrated by a lack of apparent GI side effects. In some embodiments, sustained release of active agent can result in a lower frequency of dosing. In some embodiments, provided formulations can show improved pharmacokinetics versus immediate release versions of the same active agent. In some embodiments, provided formulations afford diffusion-controlled release kinetics over an approximate 10-hour period. In some embodiments, an active agent release profile approximates diffusion-controlled release kinetics.

In some embodiments, provided compositions include active agent in an amount that is at least 3-fold greater than that of carbomer homopolymer. In some embodiments, provided compositions include active agent in an amount that is at least 6-fold greater than that of carbomer homopolymer. In some embodiments, provided compositions include active agent in an amount that is at least 10-fold greater than that of carbomer homopolymer. In some embodiments, provided compositions include active agent in an amount that is at least 20-fold greater than that of carbomer homopolymer. In some embodiments, provided compositions include active agent in an amount that is at least 40-fold greater than that of carbomer homopolymer.

Problems with Acamprosate Compositions

As disclosed above and in the Prior Fogel Filings, acamprosate is useful for the treatment of neuropsychiatric disorders, including tardive dyskinesia and other movement disorders induced by chronic exposure of patients to neuroleptic (antipsychotic) drugs, Tourette syndrome, and mental disorders such as posttraumatic stress disorder (PTSD) and obsessive-compulsive disorder (OCD), due to its actions on glutamate and GABA transmission. In addition, acamprosate may be useful in the treatment of Psychogenic Nonepileptic Seizures (PNES). Among other things, the Prior Fogel Filings specifically demonstrated that homopolymer type B carbomers and homopolymer type A carbomers can be particularly useful when preparing high dose formulations, and particularly when preparing tablet formulations containing a high dose of active agent (e.g., tablet formulations in which a significant percentage of the weight of the tablet is comprised of active agent).

As disclosed above, high dose compositions pose particular challenges for formulation. Active agents typically do not have appropriate characteristics to form a viable tablet, and there is limited physical space to include other components, if the final composition is to be small enough in size (e.g., to be easily swallowed or to be a mini-tablet). Furthermore, high dose compositions are often accompanied by unwanted side effects (e.g., due to dose dumping and/or negative food effects). Providing an extended-release high dose composition can overcome some of these side effects but also poses its own challenges, including identification of components that can be added in suitable quantities (e.g., in order for the tablet to be a suitable size) and that impart acceptable tablet characteristics (e.g., friability, hardness, etc.). The Prior Fogel Filings demonstrated that acamprosate calcium could be formulated as an extended release high dose tablet with the addition of low weight percentages of homopolymer type B carbomers or homopolymer type A carbomers.

The present disclosure identifies the source of a problem with existing acamprosate tablet compositions and/or with technologies used to prepare them, wherein the tablet compositions fail to achieve one or more desirable characteristics. Desirable tablet characteristics can include any of the following characteristics alone or in combination:
(i) extended release dissolution profile;
(ii) dissolution profile that is substantially the same at pH 1.0 and at pH 4.5;
(iii) dissolution profile that is substantially the same as a reference tablet (e.g., Formulation 2);
(iv) suitable friability;
(v) suitable hardness;
(vi) suitable firmness according to the scale of Example 2;
(vii) suitable % swelling by length;
(viii) suitable % swelling by width;
(ix) suitable % erosion;
(x) suitable shape; and
(xi) suitable size.
   The present disclosure also provides novel compositions and technologies that achieve tablet compositions with desirable characteristics and presents solutions to the above-identified problem.

Without wishing to be bound by any particular theory, the present disclosure proposes that dissolution rates achieved with the provided formulations are unique and well-suited to the administration of high dose active agents. In some embodiments, greater than 90% of active agent is released over 12 hours at pH 1.0. In some embodiments, greater than 90% of active agent is released over 12 hours at pH 4.5. In some embodiments, the release rate of active agent is linear with the square root of time. In some embodiments, greater than 70% of active agent is released over 6 hours at pH 1.0. In some embodiments, greater than 70% of active agent is released over 6 hours at pH 4.5. In some embodiments, the release of active agent is accomplished without degradation of the tablet composition.

Alternatively or additionally, without wishing to be bound by any particular theory, the present disclosure proposes that the release characteristics of the formulations of the present disclosure may be a relevant feature. In some embodiments, release characteristics of tablet compositions of provided formulations are comparable at pH values that are representative of the fed and fasted state in a subject. In some embodiments, provided formulations have comparable release characteristics in both 0.1 N HCl solution (pH 1.0, a pH value representative of a fasted subject) and in acetate buffer (pH 4.5, a pH value representative of a fed subject). In some embodiments, provided formulations have comparable release profiles of an active agent at pH 1.0 and pH 4.5.

Without wishing to be bound by theory, comparable release profiles at pH 1.0 and pH 4.5 is a relevant feature, because such compositions may be taken on an empty stomach (fasted) or with a meal (fed) and achieve similar release of active agent, which may lead to better patient compliance. Accordingly, in some embodiments, compositions with comparable release profiles in both fed and fasted states can be administered without regard to the fed or fasted status of the patient.

Alternatively or additionally, without wishing to be bound by any particular theory, the present disclosure proposes that the release characteristics of the Prior Fogel Formulations may be desirable. A similarity factor (f2) of the release profile of a provided tablet composition relative to the release profile of a reference tablet composition (e.g., a tablet composition of the Prior Fogel Formulations) is calculated as described in Example 5. A desirable similarity factor (f2) can be >40%, >50%, >60%, >70%, >80%, or >90%. A desirable similarity factor (f2) can be in a range of about 40% to about 100%, about 50% to about 100%, about 60% to about 100%, about 70% to about 100%, about 80% to about 100%, or about 90% to about 100%.

Alternatively or additionally, without wishing to be bound by any particular theory, the present disclosure proposes that friability achieved with provided tablet compositions is unique and well suited to administration of high dose active agents. Friability refers to the property of a composition to maintain (or lose) structural integrity. Those of ordinary skill in the art are aware that satisfactory tablet compositions are characterized by friability within a desirable range. Friability testing is typically performed to assess likelihood of a tablet breaking into smaller pieces under relevant conditions (e.g., during manufacturing, storage, transit, etc.). Friability values are typically expressed as a % of the tablet remaining after application of relevant conditions or stresses. Tablets which have a poor friability value crumble easily and are typically understood to not be viable as a commercial product (e.g., due to poor stability). Typical friability tests evaluate ability of a tablet to withstand abrasion in packaging, handling and shipping. For example, a weight (w1) may be determined for tablets at an initial time, prior to application of a relevant condition or stress. Subsequently, a tablet is exposed to the condition or stress, typically by being placed in a friabilator, where they are exposed to rotation at a selected speed and for a selected amount of time. A second weight (w2) in then determined, and is compared with the initial weight. Friability, which is expressed as a percent, is then calculated according to the formula: Friability=(w1−w2)×100/w1. In some embodiments, desirable tablets are typically characterized by a friability of <3%, <2%, <1%, or <0.5%. In some embodiments, desirable tablets are typically characterized by a friability in the range of 0% to about 3%, 0% to about 2%, 0% to about 1%, or 0% to about 0.5%. In some embodiments, a tablet composition has a friability value of <3%. In some embodiments, a tablet composition has a friability value of <2%. In some embodiments, a tablet composition has a friability value of <1%. In some embodiments, a tablet composition has a friability value of <0.5%.

Alternatively or additionally, without wishing to be bound by any particular theory, the present disclosure proposes that tablet compositions described herein achieve a tablet hardness that renders them superior to certain other comparable tablet compositions. Acceptable hardness values can be >3 kp, >5 kp, >10 kp, >15 kp, or >18 kp. Acceptable hardness values can be in the range of about 3 kp to about 20 kp, about 5 kp to about 20 kp, about 10 kp to about 20 kp, about 15 kp to about 20 kp, or about 18 kp to about 20 kp.

Alternatively or additionally, without wishing to be bound by any particular theory, the present disclosure proposes that tablet compositions described herein achieve a tablet firmness that renders them superior to certain other comparable tablet compositions. Tablet firmness can be assessed on a 1-5 scale, as described in Example 2. In some embodiments, acceptable firmness values are in the range of about 3.0 to about 5.0, about 4.0 to about 5.0, or about 4.5 to about 5.0. In some embodiments, acceptable firmness values are >3.0, >4.0, or >4.5.

Alternatively or additionally, without wishing to be bound by any particular theory, the present disclosure proposes that tablet compositions described herein achieve a % swelling by length at pH 1.0, pH 4.5, and/or pH 6.8 that renders them superior to certain other comparable tablet compositions. In some embodiments, acceptable % swelling (length) values are >100%, >110%, >120%, >130%, >140%, >150%, >160%, >170%, >180%, or >190%. In some embodiments, acceptable % swelling (length) values are in the range of about 100% to about 110%, about 100% to about 120%, about 100% to about 130%, about 100% to about 140%, about 100% to about 150%, about 100% to about 160%, about 100% to about 170%, about 100% to about 180%, about 130% to about 140%, about 130% to about 160%, or about 130% to about 180%.

In some embodiments, a tablet composition has a % swelling (length) in the range of about 100% to about 110% in 0.1 N HCl (pH 1.0). In some embodiments, a tablet composition has a % swelling (length) in the range of about 110% to about 120% in 0.1 N HCl (pH 1.0). In some embodiments, a tablet composition has a % swelling (length) in the range of about 110% to about 130% in 0.1 N HCl (pH 1.0). In some embodiments, a tablet composition has a swelling (length) in the range of about 130% to about 180% in 0.1 N HCl (pH 1.0).

In some embodiments, a tablet composition has a % swelling (length) in the range of about 100% to about 110% in acetate buffer (pH 4.5). In some embodiments, a tablet composition has a % swelling (length) in the range of about 110% to about 120% in acetate buffer (pH 4.5). In some embodiments, a tablet composition has a % swelling (length) in the range of about 110% to about 130% in acetate buffer (pH 4.5). In some embodiments, a tablet composition has a % swelling (length) in the range of about 130% to about 180% in acetate buffer (pH 4.5).

In some embodiments, a tablet composition has a % swelling (length) in the range of about 100% to about 110% in phosphate buffer (pH 6.8). In some embodiments, a tablet composition has a % swelling (length) in the range of about 110% to about 120% in phosphate buffer (pH 6.8). In some embodiments, a tablet composition has a % swelling (length) in the range of about 110% to about 130% in phosphate buffer (pH 6.8). In some embodiments, a tablet composition has a % swelling (length) in the range of about 130% to about 180% in phosphate buffer (pH 6.8).

Alternatively or additionally, without wishing to be bound by any particular theory, the present disclosure proposes that tablet compositions described herein achieve a % swelling by width at pH 1.0, pH 4.5, and/or pH 6.8 that renders them superior to certain other comparable tablet compositions. In some embodiments, acceptable % swelling (width) values are >100%, >110%, >120%, >130%, >140%, >150%, >160%, >170%, >180%, or >190%. In some embodiments, acceptable % swelling (width) values are in the range of about 100% to about 110%, about 100% to about 120%, about 100% to about 130%, about 100% to about 140%, about 100% to about 150%, about 100% to about 160%, about 100% to about 170%, about 100% to about 180%, about 130% to about 140%, about 130% to about 160%, or about 130% to about 180%.

In some embodiments, a tablet composition has a % swelling (width) in the range of about 130% to about 140% in 0.1 N HCl (pH 1.0). In some embodiments, a tablet composition has a % swelling (width) in the range of about 130% to about 160% in 0.1 N HCl (pH 1.0). In some embodiments, a tablet composition has a % swelling (width) in the range of about 130% to about 180% in 0.1 N HCl (pH 1.0).

In some embodiments, a tablet composition has a % swelling (width) in the range of about 130% to about 140% in acetate buffer (pH 4.5). In some embodiments, a tablet composition has a % swelling (width) in the range of about 130% to about 160% in acetate buffer (pH 4.5). In some embodiments, a tablet composition has a % swelling (width) in the range of about 130% to about 180% in acetate buffer (pH 4.5).

In some embodiments, a tablet composition has a % swelling (width) in the range of about 130% to about 140% in phosphate buffer (pH 6.8). In some embodiments, a tablet composition has a % swelling (width) in the range of about 130% to about 160% in phosphate buffer (pH 6.8). In some embodiments, a tablet composition has a % swelling (width) in the range of about 130% to about 180% in phosphate buffer (pH 6.8).

Alternatively or additionally, without wishing to be bound by any particular theory, the present disclosure proposes that tablet compositions described herein achieve a erosion at pH 1.0, pH 4.5, and/or pH 6.8 after 2 and/or 6 hours that renders them superior to certain other comparable tablet compositions. % Erosion can be measured as described in Example 5. In some embodiments, desirable % erosion values are ≤30%, ≤40%, ≤50%, ≤60%, ≤70%, or ≤80%. In some embodiments, desirable % erosion values are in the range of about 20% to about 30%, about 20% to about 40%, about 20% to about 50%, about 20% to about 60%, about 20% to about 70%, about 20% to about 80%, about 30% to about 50%, or about 30% to about 60%. In some embodiments, a tablet composition has a % erosion value after 2 h in the range of about 20% to about 50%. In some embodiments, a tablet composition has a % erosion value after 6 h in the range of about 20% to about 80%.

In some embodiments, a tablet composition has a % erosion value at pH 1.0 in the range of about 30% to about 40%. In some embodiments, a tablet composition has a erosion value at pH 1.0 in the range of about 40% to about 50%. In some embodiments, a tablet composition has a % erosion value at pH 1.0 in the range of about 50% to about 60%.

In some embodiments, a tablet composition has a % erosion value at pH 4.5 in the range of about 30% to about 40%. In some embodiments, a tablet composition has a % erosion value at pH 4.5 in the range of about 40% to about 50%. In some embodiments, a tablet composition has a % erosion value at pH 4.5 in the range of about 50% to about 60%.

In some embodiments, a tablet composition has a % erosion value at pH 6.8 in the range of about 30% to about 40%. In some embodiments, a tablet composition has a % erosion value at pH 6.8 in the range of about 40% to about 50%. In some embodiments, a tablet composition has a % erosion value at pH 6.8 in the range of about 50% to about 60%.

Alternatively or additionally, without wishing to be bound by any particular theory, the present disclosure proposes that tablet compositions described herein with a particular size and/or shape render them superior to certain other comparable tablet compositions. Difficulty swallowing tablets can be a problem for many individuals. In some cases, this can lead to adverse events and patient noncompliance with treatment regimens. Surveys have shown that problems with swallowing tablets and capsules goes beyond the patient population with clinically recognized dysphagia and may affect as much as 40% of the American population. Of those who reported difficulty swallowing tablets and capsules, less than a quarter discussed the problem with their health care professional, 8% admitted to skipping a dose of prescribed medication, and 4% have discontinued therapy because the tablets and/or capsules were difficult to swallow (Harris Interactive Inc. for Schwarz Pharma, 2003, Pill-Swallowing Problems in America: A National Survey of Adults. 1-39). Those who report difficulty swallowing tablets frequently mention the size of the tablet being a major factor (Bhosle M, Benner J, DeKoven M, Shelton J., 2009, Difficult to Swallow: Patient Preferences for Alternative Valproate Pharmaceutical Formulations. Patient Prefer Adherence 3, 161-171; Agency for Health Care Policy and Research, March 1999, Diagnosis and Treatment of Swallowing Disorders (Dysphagia) in Acute-Care Stroke Patients. Summary, Evidence Report/Technology Assessment: Number 8). Accordingly, compliance with a medication regimen may be influenced by tablet size.

Studies have been performed in a cohort of frail elderly persons to examine the effect of the size of tablets on the status of swallowing during taking medicine. The group determined a desirable size of tablet using subjective evaluation. The most desired size based upon ease of swallowing and handling is in the range of about 7 mm to about 8 mm as the largest dimension, though tablets and capsules of up to 22 mm in the largest dimension are considered acceptable by the FDA. Other studies have shown that increases in size are associated with increases in patient complaints about swallowing, and in particular when tablets are of a size greater than approximately 8 mm in diameter. In another study measuring time of esophageal transit, significantly more patients were aware of the tablets sticking in the esophagus when the tablet was >8 mm in the largest dimension. Prolonged esophageal transit time can lead to pain and localized esophagitis due to disintegration of the tablet in the esophagus. This can further lead to serious medical conditions such as ulceration, stricture, and perforation.

Shape also plays a role in the ease of a patient swallowing medication. Additionally, the shape of a tablet may have an impact on the esophageal transit time. For any given size of a tablet, certain shapes may be easier to swallow than others. In vitro studies have shown that flat tablets have a greater adherence to the esophagus than capsule-shaped tablets (e.g., oval-shaped) (Marvola M., Rajaniemi M., Marttila E., Vahervuo K., Sothmann A., 1983, Effect of Dosage Form and Formulation Factors on the Adherence of Drugs to the Esophagus. Journal of Pharmaceutical Sciences 72(9), 1034-1036). Human studies have suggested that oval tablets may be easier to swallow and have faster esophageal transit times than the corresponding round tablets of the same weight (Channer, K, Virjee, J P. 1986, The Effect of Size and Shape of Tablets on their Esophageal Transit. Journal of Clinical Pharmacology, 26, 141-146; Hey H., Jorgensen F., Sorensen K., Hasselbelch H., Wamberg T., 1982, Esophageal Transit of Six Commonly used Tablets and Capsules. British Medical Journal 285, 1717-1719). Accordingly, compliance with a medication regimen may be influenced by the tablet shape.

The present disclosure provides tablet compositions of a size and shape so as to facilitate swallowing and/or to facilitate patient compliance with a medication regimen. In some embodiments, acceptable sizes of tablet compositions are ≤22 mm, ≤20 mm, ≤18 mm, ≤15 mm, ≤13 mm, ≤10 mm, ≤8 mm, ≤6 mm, ≤4 mm, or ≤2 mm in the largest dimension. In some embodiments, acceptable lengths for the largest dimension of a tablet composition are in the range of about 1 mm to about 22 mm, about 2 mm to about 22 mm, about 4 mm to about 22 mm, about 8 mm to about 22 mm, about 18 mm to about 22 mm, about 4 mm to about 20 mm, about 8 mm to about 20 mm, about 15 mm to about 20 mm, about 1 mm to about 8 mm, about 2 mm to about 8 mm, about 4 mm to about 8 mm, about 1 mm to about 4 mm, or about 2 mm to about 4 mm.

In some embodiments, acceptable shapes of tablet compositions are oval or round. In some embodiments, a tablet composition is oval. In some embodiments, a tablet composition is round.

### Dosage Forms of Provided Compositions

### Tablet Compositions

In some embodiments, provided compositions are in an oral dosage form, such as a pill, tablet, capsule, or other composition that can be swallowed by a patient. In some embodiments, provided compositions are in the form of beads, pellets, spheroids, sprinkles, microspheres, microparticles, mini-tablets, etc. In some embodiments, provided compositions are tablet compositions. In some embodiments, tablet compositions comprise a formulation described herein. In some embodiments, an oral dosage form comprises a single tablet composition (e.g., a tablet). In some embodiments, an oral dosage form comprises a plurality of tablet compositions (e.g., mini-tablets or sprinkles).

In some embodiments, provided compositions are in the form of a coated or uncoated tablet composition. Many coatings for tablets are generally known in the art, and any suitable coating can be utilized. In some embodiments, a coated composition is an oval-shaped tablet. In some embodiments, a coated composition is a round or circular tablet. In some embodiments, a coating is Opadry® II White or Opadry® II Blue. In some embodiments, the total weight of the coating is between about 2% and about 4% of the weight of the uncoated tablet composition. In some embodiments, the coating does not affect release characteristics (e.g., rate and/or kinetics of release) of a provided composition.

In some embodiments, acceptable sizes of tablet compositions comprising a single tablet are ≤22 mm, ≤20 mm, ≤18 mm, ≤15 mm, ≤13 mm, ≤10 mm, ≤8 mm, ≤6 mm, ≤4 mm, or ≤2 mm in the largest dimension. In some embodiments, acceptable lengths for the largest dimension of a tablet composition are in the range of about 1 mm to about 22 mm, about 2 mm to about 22 mm, about 4 mm to about 22 mm, about 8 mm to about 22 mm, about 18 mm to about 22 mm, about 4 mm to about 20 mm, about 8 mm to about 20 mm, about 15 mm to about 20 mm, about 1 mm to about 8 mm, about 2 mm to about 8 mm, about 4 mm to about 8 mm, about 1 mm to about 4 mm, or about 2 mm to about 4 mm.

### Mini-Tablets

The present disclosure includes the recognition that it may be desirable to provide formulations that are small in size but provide a high dose of an active agent. In some patient populations, there can be a lesser ability to swallow tablets. For example, children and adolescents, in addition to the elderly, are more likely to have difficulties swallowing tablets. In such patient populations, a composition comprising a multitude of small tablets (i.e., mini-tablets) with a high dose of active agent may be effective in increasing patient compliance.

The present disclosure thus provides insight that oral dosage forms comprising small tablets (i.e., mini-tablets) that comprise and/or deliver acamprosate (e.g., in a pharmaceutically acceptable salt form) are particularly desirable and/or useful (for example, with populations that may struggle to swallow large tablets and/or where particularly high doses are desirable). The present disclosure provides such mini-tablet compositions that comprise and/or deliver acamprosate. In some embodiments, mini-tablet compositions are mini-tablets comprising formulations described herein. However, provided insights relating to mini-tablet dosage forms, and uses thereof may, in some embodiments, be applicable to other acamprosate compositions (e.g., Prior Fogel Formulations, and/or one or more other formulations, e.g., as described in one or more of WO 2012/050922, U.S. Pat. No. 6,426,087, or “CAMPRAL® (acamprosate calcium) Delayed-Release Tablets, Highlights of Prescribing Information,” 2004.

In some embodiments, mini-tablets can be dispersed in suitable food, such as applesauce or pudding, so that patients who have trouble swallowing can take the medication in a different way. By formulating the active agent in a smaller format, swallowing is facilitated, which may benefit certain populations, including children, adolescents, and the elderly, that have difficulty swallowing tablets to comply with a medication regimen.

In some embodiments, oral dosage forms comprising mini-tablets as described herein are provided as beads (e.g., mini-tablets) in a capsule. In some embodiments, a capsule comprising mini-tablets of provided formulations can be broken and the internal mini-tablets can be sprinkled onto soft foods to allow for swallowing without chewing.

In some embodiments, the largest dimension of a provided mini-tablet is ≤4 mm, ≤3 mm, ≤2 mm, or ≤1 mm. In some embodiments, the largest dimension of a provided mini-tablet is in the range of from about 4 mm to about 0.5 mm, from about 4 mm to about 1 mm, from about 4 mm to about 2 mm, from about 3 mm to about 0.5 mm, from about 3 mm to about 1 mm, from about 3 mm to about 2 mm, from about 2 mm to about 0.5 mm, or from about 2 mm to about 1 mm.

In some embodiments, each mini-tablet may be individually and independently coated or uncoated. In some embodiments, a capsule comprising multiple mini-tablets may be coated or uncoated. Many coatings for tablets are generally known in the art, and any suitable coating can be utilized.

In some embodiments, oral dosage forms comprising a plurality of mini-tablets as described herein are formulated (e.g., in a capsule) so that the total dose of acamprosate in the dosage form is about 400 mg, about 500 mg, about 600 mg, about 700 mg, about 800 mg, about 900 mg, or about 1000 mg. In some such embodiments, acamprosate is in a dose of about 800 mg. In some embodiments, acamprosate is in a dose of about 400 mg.

In some embodiments, oral dosage forms comprising a plurality of mini-tablets as described herein are formulated (e.g., in a capsule) so that the total dose of acamprosate (i.e., the dose per unit dosage form) is at least 400 mg, 500 mg, 600 mg, 700 mg, 800 mg, 900 mg, or 1000 mg. In some such embodiments, the dose of acamprosate is at least 400 mg. In some embodiments, the dose of acamprosate is at least 800 mg.

### Components of Provided Tablet Compositions

As noted above, the formulation of drug products containing a large dose of active pharmaceutical ingredient (“API”), is challenging, particularly when preparing an oral formulation. The Prior Fogel Filings specifically demonstrated that homopolymer type B carbomers and homopolymer type A carbomers can be particularly useful when preparing high dose formulations, and particularly when preparing tablet formulations containing a high dose of active agent. Furthermore, the Prior Fogel Formulations demonstrated certain desirable characteristics, including an extended release profile and fed/fasted equivalence.

The present disclosure provides different compositions for high dose formulations of acamprosate, which also demonstrate certain desirable characteristics (see above). In some embodiments, the provided compositions utilize different amounts of carbomer homopolymers and/or alternative carbomer homopolymers (e.g., carbomer homopolymer C). In some embodiments, the provided compositions outperform the Prior Fogel Formulations with respect to one or more desirable characteristics.

In some embodiments, the total weight of a provided composition is in the range of about 900 mg to about 1300 mg, about 1000 mg to about 1280 mg, about 1120 mg to about 1200 mg, or about 1140 mg to about 1180 mg. In some embodiments, the total weight of an uncoated tablet composition is in the range of about 900 mg to about 1300 mg, about 1000 mg to about 1280 mg, about 1120 mg to about 1200 mg, about 1140 mg to about 1180 mg, about 1080 mg to about 1160 mg, or about 1100 mg to about 1140 mg. In some embodiments, the total weight of an uncoated tablet composition is in the range of about 1140 mg to about 1180 mg. In some embodiments, the total weight of an uncoated tablet composition is in the range of about 1100 mg to about 1140 mg.

In some embodiments, the total weight of a provided composition is about 900 mg, about 950 mg, about 1000 mg, about 1050 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1250 mg, or about 1300 mg. In some embodiments, the total weight of an uncoated tablet composition is about 900 mg, about 950 mg, about 1000 mg, about 1050 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1250 mg, or about 1300 mg. In some embodiments, the total weight of an uncoated tablet composition is about 1120 mg. In some embodiments, the total weight of an uncoated tablet composition is about 1160 mg.

In some embodiments, provided compositions comprise acamprosate as the active agent. In some embodiments, in a provided composition, acamprosate is in a pharmaceutically acceptable salt form, such as acamprosate calcium. In some embodiments, a provided uncoated tablet composition comprises acamprosate in a pharmaceutically acceptable salt form. In some embodiments, a provided uncoated tablet composition comprises acamprosate calcium.

In some embodiments, a provided tablet composition comprises a dose of acamprosate in the range of about 30 wt % to about 80 wt %, about 40 wt % to about 80 wt %, about 50 wt % to about 80 wt %, about 60 wt % to about 80 wt %, about 30 wt % to about 50 wt %, about 40 wt % to about 60 wt %, about 50 wt % to about 70 wt %, or about 60 wt % to about 80 wt %. In some embodiments, acamprosate is in a dose in a range of about 60 wt % to about 80 wt %. In some embodiments, acamprosate is in a dose in a range of about 30 wt % to about 50 wt %. In some embodiments, acamprosate is in a dose in a range of about 66 wt % to about 72 wt %. In some embodiments, acamprosate is in a dose in a range of about 36 wt % to about 44 wt %.

In some embodiments, a provided tablet composition comprises a dose of acamprosate of about 30 wt %, about 40 wt %, about 50 wt %, about 60 wt %, about 70 wt %, or about 80 wt %. In some embodiments, acamprosate is in a dose of about 70 wt %. In some embodiments, acamprosate is in a dose of about 60 wt %. In some embodiments, acamprosate is in a dose of about 40 wt %. In some embodiments, acamprosate is in a dose of about 71 wt %. In some embodiments, acamprosate is in a dose of about 67 wt %.

In some embodiments, a provided tablet composition comprises a dose of acamprosate of about 400 mg, about 500 mg, about 600 mg, about 700 mg, about 800 mg, about 900 mg, or about 1000 mg. In some embodiments, acamprosate is in a dose of about 800 mg. In some embodiments, acamprosate is in a dose of about 400 mg.

In some embodiments, provided tablet compositions comprise a dose of acamprosate that is at least 400 mg, at least 500 mg, at least 600 mg, at least 700 mg, at least 800 mg, at least 900 mg, or at least 1000 mg. In some embodiments, the dose of acamprosate is at least 400 mg. In some embodiments, the dose of acamprosate is at least 800 mg.

In some embodiments, provided compositions comprise one or more carbomer homopolymers. Carbomer homopolymers are high molecular weight polymers of acrylic acid that are chemically cross-linked with polyalkenyl alcohols or divinyl glycol. Carbomer homopolymers are divided into three types: A, B, and C. Carbomer homopolymers are polymers of acrylic acid chemically cross-linked with allyl ethers of pentaerythritol. All three carbomer homopolymer types are flocculated powders averaging about 2 to 7 microns in diameter. The three types of carbomer homopolymer are differentiated by their viscosity characteristics. The aqueous viscosity for a 0.5% aqueous solution of carbomer homopolymer type A is 4,000-11,000 mPa·s, for type B, 25,000-45,000 mPa·s and for type C, 40,000-60,000 mPa·s.

The molecular weights of carbomer homopolymers are extremely high due to extensive cross-linking. Each particle can be viewed as a network structure of polymer chains that are interconnected by crosslinks. Because of their large molecular weights and high degree of cross-linking, carbomer homopolymers do not dissolve in water but instead swell in water up to 1,000 times their original volume and ten times their original diameter. Common analytical techniques used to characterize the molecular weight of linear (i.e., not cross linked) polymers such as gel permeation chromatography, light scattering, ultracentrifugation and osmometry all require that the polymer be soluble and are therefore not useful for determining the molecular weight of a carbomer homopolymer. The calculated molecular weight for a crosslinked carbomer homopolymer with a primary particle size of 0.2 micron could be as high as 4.5 billion due to the cross linking of many polymer chains.

Typical usage levels of carbomer homopolymers in extended-release tablets are 3-30% w/w depending on the properties of the active agent, co-excipients and processing parameters. The appropriate amount of carbomer required for extended release is a function of the properties of the active agent, other additives in the formulation and the processing parameters. Generally, increasing the amount of carbomer in a formulation leads to slower and more linear release of the active agent. Commonly, higher doses of active agent, especially active agents with good water solubility, require larger amounts of polymer. For water-soluble active agents, 10% w/w carbomer homopolymer is recommended when used in a wet granulation process. For a direct compression process, 25% w/w carbomer homopolymer is recommended. For active agents that have low water solubility, less carbomer is required.

The present disclosure details the surprising finding that certain anionic and higher solubility active agents (e.g., acamprosate) can be successfully formulated for extended release using lower than typical amounts of carbomer homopolymer (e.g., 2 wt % to 12 wt %). Such insight leads to the ability to utilize carbomer homopolymers in tablet compositions even when active agent is present at such a high dose that it comprises a material percentage of the tablet weight (e.g., at least 40 wt %, at least 50 wt %, at least 60 wt %, at least 70 wt %, at least 80 wt %, or more of the tablet weight). Furthermore, utilization of smaller amounts of carbomer homopolymer than are typically recommended allows for extended release tablet compositions of suitable size (e.g., tablet compositions that are easy to swallow).

Without wishing to be bound by theory, carbomer homopolymers are anionic in nature, and therefore, the release of active agent may be pH-dependent. Accordingly, carbomer homopolymers have typically been paired with cationic active agents (e.g., basic active agents in a salt form). The present disclosure details the surprising result that carbomer homopolymers can be particularly useful with an anionic active agent, notwithstanding their own anionic characteristics.

In some embodiments, provided tablet compositions comprise a carbomer homopolymer, such as carbomer homopolymer type A, carbomer homopolymer type B, or carbomer homopolymer type C. In some embodiments, a tablet composition comprises carbomer homopolymer type A. In some embodiments, a tablet composition comprises carbomer homopolymer type B. In some embodiments, a tablet composition comprises carbomer homopolymer type C.

In some embodiments, tablet compositions provided by the present disclosure include a polymer matrix that comprises or consists of a carbomer homopolymer. In some such embodiments, a carbomer homopolymer is a carbomer homopolymer type A. In some embodiments, a carbomer homopolymer is a carbomer homopolymer type B. In some embodiments, a carbomer homopolymer is a carbomer homopolymer type C.

In some embodiments, a provided tablet composition comprises a carbomer homopolymer in the range of about 2 wt % to about 15 wt %, about 4 wt % to about 12 wt %, about 6 wt % to about 10 wt %, about 3 wt % to about 7 wt %, or about 4 wt % to about 6 wt %. In some embodiments, a carbomer homopolymer is present in an amount of about 2 wt % to about 15 wt %. In some embodiments, a carbomer homopolymer is present in an amount of about 3 wt % to about 7 wt %. In some embodiments, a carbomer homopolymer is present in an amount of about 4 wt % to about 6 wt %.

In some embodiments, a carbomer homopolymer is present in an amount of about 2 wt %, about 3 wt %, about 4 wt %, about 5 wt %, about 6 wt %, about 7 wt %, about 8 wt %, about 9 wt %, about 10 wt %, about 11 wt %, or about 12 wt %. In some embodiments, a carbomer homopolymer is present in an amount of about 3 wt %. In some embodiments, a carbomer homopolymer is present in an amount of about 5 wt %. In some embodiments, a carbomer homopolymer is present in an amount of about 8 wt %. In some embodiments, a carbomer homopolymer is present in an amount of about 13 wt %.

In some embodiments, a provided tablet composition comprises a carbomer homopolymer in a range of about 30 mg to about 140 mg, about 50 mg to about 120 mg, about 70 mg to about 100 mg, about 30 mg to about 70 mg, about 50 mg to about 100 mg, about 50 mg to about 70 mg, or about 70 mg to about 100 mg. In some embodiments, a carbomer homopolymer is in an amount of about 50 mg to about 70 mg. In some embodiments, a carbomer homopolymer is in an amount of about 70 mg to about 100 mg.

In some embodiments, a carbomer homopolymer is in an amount of about 30 mg, about 40 mg, about 50 mg, about 60 mg, about 70 mg, about 80 mg, about 90 mg, about 100 mg, about 110 mg, about 120 mg, about 130 mg, or about 140 mg. In some embodiments, a carbomer homopolymer is present in an amount of about 30 mg. In some embodiments, a carbomer homopolymer is present in an amount of about 60 mg. In some embodiments, a carbomer homopolymer is present in an amount of about 90 mg. In some embodiments, a carbomer homopolymer is present in an amount of about 140 mg.

In some embodiments, a provided composition comprises a carbomer homopolymer type A in a range of about 2 wt % to about 15 wt %, about 4 wt % to about 12 wt %, about 6 wt % to about 10 wt %, about 3 wt % to about 7 wt %, or about 4 wt % to about 6 wt %. In some embodiments, a carbomer homopolymer type A is present in about 2 wt % to about 15 wt %. In some embodiments, a carbomer homopolymer type A is in an amount of about 3 wt % to about 7 wt %. In some embodiments, a carbomer homopolymer type A is in an amount of about 4 wt % to about 6 wt %. In some embodiments, a carbomer homopolymer type A is in an amount of about 4 wt % to about 6 wt %.

In some embodiments, a carbomer homopolymer type A is in an amount of about 2 wt %, about 3 wt %, about 4 wt %, about 5 wt %, about 6 wt %, about 7 wt %, about 8 wt %, about 9 wt %, about 10 wt %, about 11 wt %, or about 12 wt %. In some embodiments, a carbomer homopolymer type A is in an amount of about 3 wt %. In some embodiments, a carbomer homopolymer type A is in an amount of about 5 wt %. In some embodiments, a carbomer homopolymer type A is in an amount of about 8 wt %. In some embodiments, a carbomer homopolymer type A is in an amount of about 13 wt %.

In some embodiments, a provided composition comprises a carbomer homopolymer type A in a range of about 30 mg to about 140 mg, about 50 mg to about 120 mg, about 70 mg to about 100 mg, about 30 mg to about 70 mg, about 50 mg to about 100 mg, about 50 mg to about 70 mg, or about 70 mg to about 100 mg. In some embodiments, a carbomer homopolymer type A is in an amount of about 50 mg to about 70 mg. In some embodiments, a carbomer homopolymer type A is in an amount of about 70 mg to about 100 mg.

In some embodiments, a carbomer homopolymer type A is in an amount of about 30 mg, about 40 mg, about 50 mg, about 60 mg, about 70 mg, about 80 mg, about 90 mg, about 100 mg, about 110 mg, about 120 mg, about 130 mg, or about 140 mg. In some embodiments, a carbomer homopolymer type A is in an amount of about 60 mg. In some embodiments, a carbomer homopolymer type A is in an amount of about 80 mg.

In some embodiments, a provided composition comprises a carbomer homopolymer type B in a range of about 2 wt % to about 15 wt %, about 4 wt % to about 12 wt %, about 6 wt % to about 10 wt %, about 3 wt % to about 7 wt %, or about 4 wt % to about 6 wt %. In some embodiments, a carbomer homopolymer type B is in an amount of about 2 wt % to about 15 wt %. In some embodiments, a carbomer homopolymer type B is in an amount of about 3 wt % to about 7 wt %. In some embodiments, a carbomer homopolymer type B is in an amount of about 4 wt % to about 6 wt %.

In some embodiments, a carbomer homopolymer type B is in an amount of about 2 wt %, about 3 wt %, about 4 wt %, about 5 wt %, about 6 wt %, about 7 wt %, about 8 wt %, about 9 wt %, about 10 wt %, about 11 wt %, or about 12 wt %. In some embodiments, a carbomer homopolymer type B is in an amount of about 5 wt %. In some embodiments, a carbomer homopolymer type B is in an amount of about 8 wt %.

In some embodiments, a provided composition comprises a carbomer homopolymer type B in a range of about 30 mg to about 140 mg, about 50 mg to about 120 mg, about 70 mg to about 100 mg, about 30 mg to about 70 mg, about 50 mg to about 100 mg, about 50 mg to about 70 mg, or about 70 mg to about 100 mg. In some embodiments, a carbomer homopolymer type B is in an amount of about 50 mg to about 70 mg. In some embodiments, a carbomer homopolymer type B is in an amount of about 70 mg to about 100 mg.

In some embodiments, a carbomer homopolymer type B is in an amount of about 30 mg, about 40 mg, about 50 mg, about 60 mg, about 70 mg, about 80 mg, about 90 mg, about 100 mg, about 110 mg, about 120 mg, about 130 mg, or about 140 mg. In some embodiments, a carbomer homopolymer type B is in an amount of about 60 mg. In some embodiments, a carbomer homopolymer type B is in an amount of about 90 mg.

In some embodiments, a provided composition comprises a carbomer homopolymer type C in the range of about 2 wt % to about 15 wt %, about 4 wt % to about 12 wt %, about 6 wt % to about 10 wt %, about 3 wt % to about 7 wt %, or about 4 wt % to about 6 wt %. In some embodiments, a carbomer homopolymer type C is in an amount of about 2 wt % to about 15 wt %. In some embodiments, a carbomer homopolymer type C is in an amount of about 4 wt % to about 12 wt %.

In some embodiments, a carbomer homopolymer type C is in an amount of about 2 wt %, about 3 wt %, about 4 wt %, about 5 wt %, about 6 wt %, about 7 wt %, about 8 wt %, about 9 wt %, about 10 wt %, about 11 wt %, or about 12 wt %. In some embodiments, a carbomer homopolymer type C is in an amount of about 3 wt %. In some embodiments, a carbomer homopolymer type C is in an amount of about 5 wt %. In some embodiments, a carbomer homopolymer type C is in an amount of about 8 wt %. In some embodiments, a carbomer homopolymer type C is in an amount of about 13 wt %.

In some embodiments, a provided composition comprises a carbomer homopolymer type C in a range of about 30 mg to about 140 mg, about 50 mg to about 120 mg, about 70 mg to about 100 mg, about 30 mg to about 70 mg, about 50 mg to about 100 mg, about 50 mg to about 70 mg, or about 70 mg to about 100 mg. In some embodiments, a carbomer homopolymer type C is in an amount of about 50 mg to about 70 mg. In some embodiments, a carbomer homopolymer type C is in an amount of about 70 mg to about 100 mg.

In some embodiments, a carbomer homopolymer type C is in an amount of about 30 mg, about 40 mg, about 50 mg, about 60 mg, about 70 mg, about 80 mg, about 90 mg, about 100 mg, about 110 mg, about 120 mg, about 130 mg, or about 140 mg. In some embodiments, a carbomer homopolymer type C is in an amount of about 60 mg. In some embodiments, a carbomer homopolymer type C is in an amount of about 80 mg.

Alternatively or additionally, in some embodiments, tablet compositions provided by the present disclosure comprise a polymer selected from the group consisting of carboxymethyl cellulose (e.g., Aqualon® CMC-7HF), an acrylates copolymer (e.g., a poly(methyl acrylate-co-methyl methacrylate-co-methacrylic acid 7:3:1, such as EUDRAGIT® FS 30 D), and gelatin type B. In some embodiments, a polymer selected from the group consisting of carboxymethyl cellulose (e.g., Aqualon® CMC-7HF), an acrylates copolymer (e.g., a poly(methyl acrylate-co-methyl methacrylate-co-methacrylic acid 7:3:1, such as EUDRAGIT® FS 30 D), and gelatin type B is present in a provided tablet composition in addition to a carbomer homopolymer.

In some embodiments, a provided composition comprises carboxymethyl cellulose (e.g., Aqualon® CMC-7HF). In some embodiments, carboxymethyl cellulose is part of a polymer matrix. In some embodiments, carboxymethyl cellulose is in an amount of about 0.5 wt % to about 9 wt %, about 3 wt % to about 9 wt %, about 5 wt % to about 9 wt %, or about 6 wt % to about 8 wt %. In some embodiments, carboxymethyl cellulose is in an amount of about 5 wt % to about 9 wt %.

In some embodiments, carboxymethyl cellulose is in an amount of about 1 wt %, about 2 wt %, about 3 wt %, about 4 wt %, about 5 wt %, about 6 wt %, about 7 wt %, about 8 wt %, or about 9 wt %. In some embodiments, carboxymethyl cellulose is in an amount of about 1 wt %. In some embodiments, carboxymethyl cellulose is in an amount of about 3 wt % (e.g., 3.5 wt %). In some embodiments, carboxymethyl cellulose is in an amount of about 7 wt % (e.g., 6.8 wt %).

In some embodiments, a provided composition comprises carboxymethyl cellulose (e.g., Aqualon® CMC-7HF) in an amount of about 10 mg to about 100 mg, about 10 mg to about 40 mg, about 40 mg to about 80 mg, or about 60 mg to about 100 mg.

In some embodiments, carboxymethyl cellulose is in an amount of about 10 mg, about 20 mg, about 30 mg, about 40 mg, about 50 mg, about 60 mg, about 70 mg, about 80 mg, about 90 mg, or about 100 mg. In some embodiments, carboxymethyl cellulose is in an amount of about 10 mg. In some embodiments, carboxymethyl cellulose is in an amount of about 40 mg. In some embodiments, carboxymethyl cellulose is in an amount of about 80 mg.

In some embodiments, a provided composition comprises an acrylates copolymer (e.g., a poly(methyl acrylate-co-methyl methacrylate-co-methacrylic acid 7:3:1, such as EUDRAGIT® FS 30 D). In some embodiments, an acrylates copolymer is part of a polymer matrix. In some embodiments, an acrylates copolymer is in an amount of about 0.5 wt % to about 9 wt %, about 0.5 wt % to about 5 wt %, or about 0.5 wt % to about 1.5 wt %. In some embodiments, an acrylates copolymer is in an amount of about 0.5 wt % to about 1.5 wt %.

In some embodiments, an acrylates copolymer is in an amount of about 0.5 wt %, about 1 wt %, about 2 wt %, about 3 wt %, about 4 wt %, or about 5 wt %. In some embodiments, an acrylates copolymer is in an amount of about 0.5 wt %.

In some embodiments, a provided composition comprises an acrylates copolymer (e.g., a poly(methyl acrylate-co-methyl methacrylate-co-methacrylic acid 7:3:1, such as EUDRAGIT® FS 30 D) in an amount of about 5 mg to about 50 mg, about 5 mg to about 20 mg, or about 5 mg to about 15 mg. In some embodiments, an acrylates copolymer is in an amount of about 5 mg, about 10 mg, about 15 mg, about 20 mg, about 30 mg, about 40 mg, or about 50 mg. In some embodiments, an acrylates copolymer is in an amount of about 10 mg.

In some embodiments, a provided composition comprises gelatin type B. In some embodiments, gelatin type B is part of a polymer matrix. In some embodiments, gelatin type B is in an amount of about 0.5 wt % to about 5 wt %, about 2 wt % to about 5 wt %, or about 3 wt % to about 4 wt %. In some embodiments, gelatin type B is in an amount of about 2 wt % to about 5 wt %.

In some embodiments, gelatin type B is in an amount of about 1 wt %, about 2 wt %, about 3 wt %, about 4 wt %, or about 5 wt %. In some embodiments, gelatin type B is in an amount of about 4 wt % (e.g., 3.6 wt %).

Those skilled in the art of pharmaceutical formulation appreciate that formulations typically include one or more components other than active agent. Formulations provided by the present disclosure may include one or more such components other than the active agent(s). Any acceptable other components for therapeutic use can be used, including those described, for example, in the incorporated material of Remington: The Science and Practice of Pharmacy (2003), which is incorporated herein by reference in its entirety. Other components can refer to any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. In some embodiments, such other components are not polymers. In some embodiments, such other components are not part of a polymer matrix (e.g., throughout which one or more active agents may be distributed), even if they may be polymers (or polymeric) themselves. In some embodiments, other components may be selected from, for example, binders, carriers, color additives, diluents, disintegrants, fillers, glidants, lubricants, and combinations thereof. In some embodiments, other components may be or comprise a coating.

In some embodiments, certain provided formulations include one or more fillers, diluents or carriers. In some embodiments, a filler, diluent, or carrier is or comprises calcium carbonate DC, calcium phosphate dibasic, calcium phosphate tribasic, calcium silicate, compressible sugars, lactose, maltodextrin, mannitol, microcrystalline cellulose (e.g., Avicel® Ph 102), pregelatinized starch, sorbitol, sugar spheres, talc, talc powder, xylitol, and combinations of one or more of the same, or the like. In some embodiments, a filler, diluent, or carrier is or comprises microcrystalline cellulose. In some embodiments, a filler, diluent, or carrier is microcrystalline cellulose in combination with one or more other agents such as, for example, calcium carbonate DC, calcium phosphate dibasic, calcium phosphate tribasic, compressible sugars, lactose, maltodextrin, mannitol, pregelatinized starch, sorbitol, xylitol, and combinations of one or more of the same, or the like. In some embodiments, a filler, diluent, or carrier is or comprises talc (e.g., talc powder). In some embodiments, a filler, diluent, or carrier is or comprises a combination of microcrystalline cellulose and talc. In some embodiments, a filler, diluent, or carrier is or comprises sugar spheres.

In some embodiments, certain provided formulations include one or more glidants. Suitable glidants are known in the art and can include CarboSil®, calcium silicate, calcium stearate, colloidal silicon dioxide, corn starch, fumed silicon dioxide, magnesium carbonate, magnesium laurel sulfate, magnesium oxide, magnesium stearate (MGST), metallic stearates, silicon dioxide, starch, starch 1500, stearowet C®, zinc stearate, and combinations thereof. In some embodiments, a provided composition comprises colloidal silicon dioxide. In some embodiments, colloidal silicon dioxide is in an amount of about 0.5 wt % to about 2 wt %.

In some embodiments, certain provided formulations include one or more lubricants, for example, to improve powder flow, prevent the blend from adhering to tableting equipment and punch surfaces and provide lubrication to allow tablets to be cleanly ejected from tablet dies. Suitable lubricants are known in the art and can include calcium stearate, glyceryl behenate, hydrogenated vegetable oil, magnesium stearate, polyethylene glycol, sodium stearyl fumarate, stearic acid, and mixtures thereof. In some embodiments, a provided composition comprises magnesium stearate. In some embodiments, magnesium stearate is in an amount of about 0.5 wt % to about 2 wt %.

In some embodiments, certain provided formulations include one or more binders, for example, to impart cohesive qualities to a formulation, and thus ensure that the resulting dosage form remains intact after compaction. Suitable binder materials include, but are not limited to, microcrystalline cellulose, gelatin, sugars (including, for example, sucrose, glucose, dextrose and maltodextrin), polyethylene glycol, waxes, natural and synthetic gums, polyvinylpyrrolidone (e.g, Povidone K90), cellulosic polymers (including, for example, hydroxypropyl cellulose, hydroxypropyl methylcellulose, methyl cellulose, hydroxyethyl cellulose, and the like). In some embodiments, the binder is a povidone. In some embodiments, the povidone is Povidone K90 (CAS number 9003-39-8).

In some embodiments, certain provided formulations include one or more disintegrants, for example, to facilitate tablet disintegration after administration, and are generally starches, clays, celluloses, algins, gums or crosslinked polymers. Suitable disintegrants include, but are not limited to, alginic acid, cornstarch, croscarmellose sodium, crospovidone, crosslinked polyvinylpyrrolidone (PVP-XL), microcrystalline cellulose, pregelatinized starch, sodium starch glycolate, Starcap 500®, and Starcap 1500 ®. In some embodiments, a provided composition comprises Starcap 500®. In some embodiments, a provided composition comprises Starcap 1500®.

In some embodiments, provided formulations include a coating, for example, a film coating. Where film coatings are involved, coating preparations can include, for example, a film-forming polymer, a plasticizer, or the like. In some embodiments, the coatings include pigments and/or opacifiers. Non-limiting examples of film-forming polymers include hydroxypropyl methylcellulose, hydroxypropyl cellulose, methylcellulose, polyvinyl pyrrolidine, and starches. Non-limiting examples of plasticizers include polyethylene glycol, tributyl citrate, dibutyl sebecate, castor oil, and acetylated monoglyceride. Furthermore, non-limiting examples of pigments and opacifiers include iron oxides of various colors, lake dyes of many colors, titanium dioxide, and the like.

In some embodiments, a provided composition includes one or more color additives or colorants. The colorants can be used in amounts sufficient to distinguish dosage form strengths. Preferably, color additives approved for use in drugs are added to the commercial formulations to differentiate tablet strengths. The use of other pharmaceutically acceptable colorants and combinations thereof are encompassed by the current invention.

### Provided Technologies for Preparing Tablet Compositions

Another aspect of the present disclosure is the preparation of tablet compositions with provided technologies. The Prior Fogel Filings disclose round or circular tablet compositions prepared by providing a preparation of granules comprising acamprosate and blending the preparation of granules with extra-granular materials comprising a carbomer homopolymer. The Prior Fogel Filings do not describe a tablet composition prepared from a preparation of granules and extra-granular materials that both comprise a cellulose derivative. The Prior Fogel Filings do not describe the preparation of an oval tablet composition by providing a preparation of granules comprising acamprosate and blending the preparation of granules with extra-granular materials comprising a carbomer homopolymer. As described above, the Prior Fogel Formulations demonstrated unexpected effects of certain polymers (e.g., carbomer homopolymer type B or carbomer homopolymer type A) for preparing extended release formulations of acamprosate or a pharmaceutically acceptable salt thereof.

While the formulations prepared in the Prior Fogel Filings can provide round tablet compositions with desirable characteristics, the present disclosure identifies a source of variability with the process when the process is used to prepare oval tablet compositions (Example 4). As disclosed above, oval tablet compositions are desirable for a number of reasons, including ease of swallowing to encourage patient compliance. The present disclosure also provides a solution to the problem of variability in the process used to prepare oval tablet compositions.

A method of preparing an uncoated tablet composition comprising acamprosate in a pharmaceutically acceptable salt form, can comprise:
(i) blending a preparation of granules that comprise the acamprosate with extra-granular materials that comprise a carbomer homopolymer to form a blended material, wherein each of the granules and the extra-granular materials comprises a cellulose derivative material; and
(ii) compressing the blended material to form the uncoated tablet composition.

In some embodiments, the method further comprises first providing a preparation of granules. In some embodiments, the providing a preparation of granules comprises (a) forming wet granules and (b) drying the wet granules to form the preparation of granules. In some embodiments, forming wet granules comprises a wet granulation step (e.g., with purified water). In some embodiments, drying the wet granules comprises drying until loss on drying (LOD) is <10%, <5%, or <2%. In some embodiments, drying the wet granules comprises drying until loss on drying (LOD) is <2%. In some embodiments, the method further comprises coating the uncoated tablet composition.

Another aspect of the present disclosure is an improvement to a method of manufacturing an oval uncoated tablet composition comprising acamprosate in a pharmaceutically acceptable salt form, and a carbomer homopolymer, wherein the acamprosate is present in a dose of at least 400 mg and the composition is prepared by blending:
(1) a preparation of granules with
(2) extra-granular materials,
   the improvement that comprises including a cellulose derivative in each of (1) the preparation of granules; and (2) the extra-granular material, so that the composition comprises:
the acamprosate in a dose of about 66 wt % to about 72 wt %;
the carbomer homopolymer in an amount of about 3 wt % to about 7 wt %; and
the cellulose derivative in an amount of about 5 wt % to about 9 wt %.

In some embodiments, a preparation of granules weighs from about 900 mg to about 1100 mg. In some embodiments, a preparation of granules weighs about 1000 mg. In some embodiments, a preparation of granules makes up about 80 wt % to about 90 wt % of the uncoated tablet composition. In some embodiments, a preparation of granules makes up about 85 wt % of the uncoated tablet composition. In some embodiments, a preparation of granules makes up about 86 wt % of the uncoated tablet composition.

Alternatively or additionally, in some embodiments, extra-granular materials weigh from about 150 mg to about 170 mg. In some embodiments, extra-granular materials weigh about 160 mg. In some embodiments, extra-granular materials make up about 10 wt % to about 20 wt % of the uncoated tablet composition. In some embodiments, extra-granular materials make up about 13 wt % of the uncoated tablet composition. In some embodiments, extra-granular materials make up about 14 wt % of the uncoated tablet composition.

Alternatively or additionally, in some embodiments, a preparation of granules comprises acamprosate in an amount of about 70 wt % to about 90 wt %. In some embodiments, a preparation of granules comprises acamprosate in an amount of about 80 wt %. In some embodiments, a preparation of granules comprises acamprosate in an amount of about 70 wt % to about 90 wt %, so that an uncoated tablet composition comprises acamprosate in an amount of about 66 wt % to about 72 wt %. In some embodiments, a preparation of granules comprises acamprosate in an amount of about 80 wt %, so that an uncoated tablet composition comprises acamprosate in an amount of about 69 wt %. In some embodiments, a preparation of granules comprises acamprosate in an amount of at least 400 mg. In some embodiments, a preparation of granules comprises acamprosate in an amount of about 400 mg to about 1000 mg. In some embodiments, a preparation of granules comprises acamprosate in an amount of about 800 mg.

Alternatively or additionally, in some embodiments, a preparation of granules comprises a cellulose derivative in an amount of about 2 wt % to about 6 wt %. In some embodiments, a preparation of granules comprises a cellulose derivative in an amount of about 4 wt %. In some embodiments, a preparation of granules comprises carboxymethyl cellulose (e.g., CMC 7HF) in an amount of about 2 wt % to about 6 wt %. In some embodiments, a preparation of granules comprises carboxymethyl cellulose (e.g., CMC 7HF) in an amount of about 4 wt % (e.g., about 40 mg).

Alternatively or additionally, in some embodiments, extra-granular materials comprise a cellulose derivative in an amount of about 20 wt % to about 30 wt %. In some embodiments, extra-granular materials comprise a cellulose derivative in an amount of about 25 wt %. In some embodiments, extra-granular materials comprise carboxymethyl cellulose (e.g., CMC 7HF) in an amount of about 20 wt % to about 30 wt %. In some embodiments, extra-granular materials comprise carboxymethyl cellulose (e.g., CMC 7HF) in an amount of about 25 wt % (e.g., 40 mg).

Alternatively or additionally, in some embodiments, both a preparation of granules and extra-granular materials comprise a cellulose derivative. In some embodiments, a preparation of granules comprises about 2 wt % to about 6 wt % of carboxymethyl cellulose (e.g., CMC 7HF), and extra-granular materials comprise about 20 wt % to about 30 wt % of carboxymethyl cellulose (e.g., CMC 7HF), so that an uncoated tablet composition comprises a total of about 5 wt % to about 9 wt % of carboxymethyl cellulose (e.g., about 80 mg).

Alternatively or additionally, in some embodiments, extra-granular materials comprise a carbomer homopolymer (e.g., carbomer homopolymer type A, carbomer homopolymer type B, or carbomer homopolymer typer C). In some embodiments, extra-granular materials comprise a carbomer homopolymer in an amount of about 15 wt % to about 90 wt %. In some embodiments, extra-granular materials comprise a carbomer homopolymer in an amount of about 35 wt % to about 60 wt %. In some embodiments, extra-granular materials comprise a carbomer homopolymer in an amount of about 37.5 wt % (e.g., 60 mg).

In some embodiments, extra-granular materials comprise carbomer homopolymer type B in an amount of about 35 wt % to about 60 wt %. In some embodiments, extra-granular materials comprise carbomer homopolymer type B in an amount of about 37.5 wt %. In some embodiments, extra-granular materials comprise carbomer homopolymer type B in an amount of about 60 mg. In some embodiments, extra-granular materials comprise carbomer homopolymer type B, in an amount so that an uncoated tablet composition comprises about 3 wt % to about 7 wt % carbomer homopolymer type B.

Alternatively or additionally, in some embodiments, extra-granular materials comprise both carbomer homopolymer type B and carboxymethyl cellulose. In some embodiments, extra-granular materials comprise carbomer homopolymer type B and carboxymethyl cellulose in a ratio of 3:2. In some embodiments, extra-granular materials comprise about 60 mg carbomer homopolymer type B and about 40 mg carboxymethyl cellulose. In some embodiments, extra-granular materials comprise carbomer homopolymer type B and carboxymethyl cellulose, in amounts so that an uncoated tablet composition comprises about 3 wt % to about 7 wt % carbomer homopolymer type B and about 5 wt % to about 9 wt % carboxymethyl cellullose.

Alternatively or additionally, in some embodiments, a preparation of granules comprises an acrylates copolymer (e.g., EUDRAGIT® FS 30 D). In some embodiments, a preparation of granules comprises about 10 mg of an acrylates copolymer. In some embodiments, a preparation of granules comprises an amount of acrylates copolymer so that the acrylates copolymer is present in an uncoated tablet composition within the range of about 0.5 wt % to about 1.5 wt %. In some embodiments, a preparation of granules comprises an amount of acrylates copolymer so that the acrylates copolymer is present in an uncoated tablet composition in about 0.9 wt %.

Alternatively or additionally, in some embodiments, extra-granular materials comprise carbomer homopolymer type B and gelatin type B. In some embodiments, extra-granular materials comprise carbomer homopolymer type B and gelatin type B in a ratio of 9:4. In some embodiments, extra-granular materials comprise about 90 mg carbomer homopolymer type B and about 40 mg gelatin type B. In some embodiments, extra-granular materials comprise carbomer homopolymer type B and gelatin type B in an amount so that an uncoated tablet composition comprises carbomer homopolymer type B in an amount of about 3 wt % to about 7 wt % and gelatin type B in an amount of about 2 wt % to about 5 wt %.

Alternatively or additionally, in some embodiments, extra-granular materials comprise carbomer homopolymer type C and carboxymethyl cellulose. In some embodiments, extra-granular materials comprise carbomer homopolymer type C and carboxymethyl cellulose in a ratio of 3:2. In some embodiments, extra-granular materials comprise carbomer homopolymer type C and carboxymethyl cellulose in a ratio of 9:4. In some embodiments, extra-granular materials comprise carbomer homopolymer type C and carboxymethyl cellulose in a ratio of 3:4. In some embodiments, extra-granular materials comprise about 60 mg carbomer homopolymer type C and about 40 mg carboxymethyl cellulose. In some embodiments, extra-granular materials comprise about 90 mg carbomer homopolymer type C and about 40 mg carboxymethyl cellulose. In some embodiments, extra-granular materials comprise about 30 mg carbomer homopolymer type C and about 40 mg carboxymethyl cellulose. In some embodiments, extra-granular materials comprise about 140 mg carbomer homopolymer type C. In some embodiments, extra-granular materials comprise carbomer homopolymer type C and optionally carboxymethyl cellulose in an amount so that an uncoated tablet composition comprises carbomer homopolymer type C in an amount of about 2 wt % to about 15 wt % and carboxymethyl cellulose in an amount of 0 wt % to about 5 wt %.

In some embodiments, provided technologies (e.g., a method of preparing an uncoated tablet composition comprising acamprosate) can be used to prepare a tablet composition comprising any one of the Formulations disclosed herein (e.g., Formulations 3-6 or 9-12). The provided technologies can also be used to improve certain characteristics of a Formulation disclosed herein (e.g., by adding an amount of cellulose derivative to both a preparation of granules and extra-granular materials).

### Combinations

Compositions described herein may also include one or more additional therapeutic agents. In some embodiments, additional therapeutic agents are selected from first-generation neuroleptics (antipsychotics), second-generation neuroleptics, selective serotonin reuptake inhibitors (SSRIs), serotonin norepinephrine reuptake inhibitors (SNRIs), and the anti-nausea drug metoclopramide. Without wishing to be bound by theory, extended-release properties of provided formulations make it feasible to formulate fixed-dose combinations of acamprosate and other agents, such as those described herein. Additional benefits of a fixed dose combination of acamprosate with another therapeutic agent include increased patient compliance, increased therapeutic effects, and reduced side effects. Fixed dose combinations with first-generation neuroleptics, for example, can provide effective treatment of psychosis with a lesser risk of metabolic side effects (e.g., weight gain, glucose intolerance, and/or increased risk of atherosclerotic cardiovascular disease) than seen with second-generation neuroleptic drugs, with a lesser risk of tardive dyskinesia than seen with first-generation neuroleptic drugs given alone, and with, unexpectedly, increased relief of mental symptoms compared with first-generation neuroleptic drugs given alone.

In some embodiments, a provided composition further comprises one or more additional therapeutic agents, such as a first generation neuroleptic, a second generation neuroleptic, a selective serotonin reuptake inhibitor (SSRI) or a serotonin-norepinephrine reuptake inhibitor (SNRI). In some embodiments, a provided composition is a unit dosage form that includes, consists of, or consists essentially of both acamprosate and at least one additional therapeutic agent. In some embodiments, a composition is a multilayer (e.g., bilayer) composition wherein each layer comprises one active agent. In some embodiments, a composition is not a bilayer composition, wherein active agents are dispersed in a polymer matrix.

In some embodiments, a composition is administered to a patient who is receiving or has received one or more other medications, such as a first generation neuroleptic, a second generation neuroleptic, a selective serotonin reuptake inhibitor (SSRI), a serotonin norepinephrine reuptake inhibitor (SNRI), an anti-nausea drug, an NMDA modulator, and/or an α_{2A} adrenergic receptor agonist, or any combination thereof.

In some embodiments, a composition as described herein further comprises or is administered to a patient who is receiving or has received a second medication selected from one or more of a neuroleptic (antipsychotic) medication, a SSRI, a SNRI, an antidepressant other than an SSRI or SNRI, an anti-anxiety medication other than an SSRI or SNRI or the like, an anti-nausea drug, an NMDA modulator, and an α_{2A} adrenergic receptor agonist.

In some embodiments, a provided composition further comprises a neuroleptic medication. In some embodiments, a provided composition further comprises a first generation neuroleptic. In some embodiments, a first generation neuroleptic is selected from chlorpromazine, fluphenazine, haloperidol, loxapine, molindone, perphenazine, pimozide, thioridazine, thiothixene, and trifluoperazine, or the like.

In some embodiments, a provided composition further comprises a second generation neuroleptic. In some embodiments, a second generation neuroleptic is selected from aripiprazole, asenapine, iloperidone, lurasidone, olanzapine, paliperidone, quetiapine, risperidone, and ziprasidone, or the like.

In some embodiments, a provided composition further comprises a SSRI or SNRI. In some such embodiments, a SSRI or SNRI is selected from one or more of citalopram, desvenlafaxine, duloxetine, escitalopram, fluoxetine, fluvoxamine, milnacipran, paroxetine, sertraline, and venlafaxine, or the like.

In some embodiments, a provided composition further comprises an anti-depressant other than an SSRI or SNRI.

In some embodiments, a provided composition further comprises an anti-anxiety medication other than an SSRI or SNRI.

In some embodiments, a provided composition further comprises an anti-nausea drug. In some embodiments, an anti-nausea drug is metoclopramide.

In some embodiments, a provided composition further comprises an NMDA modulator. In some embodiments, a NMDA modulator is memantine. In some embodiments, a NMDA modulator is amantadine.

In some embodiments, a provided composition further comprises an α_{2A} adrenergic receptor agonist. In some embodiments, the α_{2A} adrenergic receptor agonist is guanfacine.

In some embodiments, a provided composition further comprises one or more additional therapeutic agents selected from the group consisting of amantadine, aripiprazole, asenapine, chlorpromazine, citalopram, clonidine, desvenlafaxine, duloxetine, escitalopram, fluoxetine, fluvoxamine, fluphenazine, guanfacine, haloperidol, iloperidone, loxapine, lurasidone, memantine, metoclopramide, milnacipran, molindone, olanzapine, paliperidone, paroxetine, perphenazine, pimozide, prazosin, quetiapine, risperidone, sertraline, thioridazine, thiothixene, trifluoperazine, venlafaxine, and ziprasidone, and any combination thereof.

In some embodiments, a provided composition is administered to a patient who is receiving or has received one or more additional therapeutic agents selected from the group consisting of amantadine, aripiprazole, asenapine, chlorpromazine, citalopram, clonidine, desvenlafaxine, duloxetine, escitalopram, fluoxetine, fluvoxamine, fluphenazine, guanfacine, haloperidol, iloperidone, loxapine, lurasidone, metoclopramide, memantine, milnacipran, molindone, olanzapine, paliperidone, paroxetine, perphenazine, pimozide, prazosin, quetiapine, risperidone, sertraline, thioridazine, thiothixene, trifluoperazine, venlafaxine, and ziprasidone, and any combination thereof.

In some embodiments, a provided composition further comprises amantadine. Amantadine (trade names Gocovri®, Symadine®, or Symmetrel®) is used to treat symptoms of Parkinson's disease, such as levodopa-induced dyskinesia (LID). Without wishing to be bound by any theory, an extended-release combination of acamprosate and amantadine may be particularly useful for treating symptoms of Parkinson's disease, such as levodopa-induced dyskinesia (LID).

In some embodiments, amantadine is in a dose of from about 50 mg to about 300 mg, such as about 50 mg, about 100 mg, about 150 mg, about 200 mg, about 250 mg, or about 300 mg, or any ranges between any two values. In some embodiments, a total daily dosage of amantadine is from about 100 mg to about 400 mg, such as about 100 mg, about 150 mg, about 200 mg, about 250 mg, about 300 mg, about 350 mg, or about 400 mg, or any ranges between any two values.

In some embodiments of a composition further comprising amantadine, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 50 mg or about 100 mg amantadine or about 1000 mg acamprosate and about 50 mg or about 100 mg amantadine.

In some embodiments, a composition comprising acamprosate and amantadine is for use in treating levodopa-induced dyskinesia in patients with Parkinson's disease. In some embodiments, a method of treating levodopa-induced dyskinesia in patients with Parkinson's disease comprises administering a therapeutically effective dose of a composition comprising acamprosate and amantadine to a patient in need thereof.

In some embodiments, a provided composition further comprises clonidine. Clonidine is used to treat high blood pressure, anxiety disorders, and attention disorders, among others. Without wishing to be bound by any theory, an extended-release combination of acamprosate and clonidine may be particularly useful for treating Tourette syndrome.

In some embodiments, clonidine is in a dose of from about 0.025 mg to about 0.25 mg, such as about 0.025 mg, about 0.05 mg, about 0.1 mg, about 0.15 mg, about 0.2 mg, or about 0.25 mg, or any ranges between any two values. In some embodiments, a total daily dosage of clonidine is from about 0.05 mg to about 0.5 mg, such as about 0.05 mg, about 0.1 mg, about 0.2 mg, about 0.3 mg, about 0.4 mg, or about 0.5 mg, or any ranges between any two values.

In some embodiments of a composition further comprising clonidine, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 0.1 mg clonidine or about 800 mg acamprosate and about 0.05 mg clonidine.

In some embodiments, a composition comprising acamprosate and clonidine is for use in treating Tourette syndrome. In some embodiments, a method of treating Tourette syndrome comprises administering a therapeutically effective dose of a composition comprising acamprosate and clonidine to a patient in need thereof.

In some embodiments, a provided composition further comprises fluvoxamine. Fluvoxamine (trade name Luvox®, among others) is used to treat obsessive-compulsive disorder (OCD), major depression disorder, and anxiety disorders, among others. Fluvoxamine is the only SSRI with sigma-1 agonist effects, and one that may be uniquely useful for treating OCD. Without wishing to be bound by any theory, an extended-release combination of acamprosate and fluvoxamine may be particularly useful for treating OCD.

In some embodiments, fluvoxamine is in a dose of from about 25 mg to about 100 mg, such as about 25 mg, about 50 mg, about 75 mg, or about 100 mg, or any ranges between any two values. In some embodiments, a total daily dosage of fluvoxamine is from about 50 mg to about 300 mg, such as about 50 mg, about 100 mg, about 150 mg, about 200 mg, about 250 mg, or about 300 mg, or any ranges between any two values.

In some embodiments of a composition further comprising fluvoxamine, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 25 mg fluvoxamine or about 800 mg acamprosate and about 75 mg fluvoxamine.

In some embodiments, a composition comprising acamprosate and fluvoxamine is for use in treating OCD. In some embodiments, a method of treating OCD comprises administering a therapeutically effective dose of a composition comprising acamprosate and fluvoxamine to a patient in need thereof.

In some embodiments, a provided composition further comprises guanfacine. Guanfacine is used to treat high blood pressure and attention disorders. Without wishing to be bound by any theory, an extended-release combination of acamprosate and guanfacine may be particularly useful for treating Tourette syndrome.

In some embodiments, guanfacine is in a dose of from about 0.25 mg to about 8 mg, such as about 0.25 mg, about 0.5 mg, about 1 mg, about 2 mg, about 4 mg, or about 8 mg, or any ranges between any two values. In some embodiments, a total daily dosage of guanfacine is from about 0.5 mg to about 10 mg, such as about 0.5 mg, about 1 mg, about 2 mg, about 4 mg, about 7 mg, or about 8 mg, or any ranges between any two values.

In some embodiments of a composition further comprising guanfacine, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 1 mg or about 4 mg guanfacine or about 1000 mg acamprosate and about 1 mg or about 4 mg guanfacine.

In some embodiments, a composition comprising acamprosate and guanfacine is for use in treating Tourette syndrome. In some embodiments, a method of treating Tourette syndrome comprises administering a therapeutically effective dose of a composition comprising acamprosate and guanfacine to a patient in need thereof.

In some embodiments, a provided composition further comprises memantine. Memantine is used to treat moderate to severe Alzheimer's disease, as well as symptoms of Parkinson's disease. In some embodiments, the present disclosure encompasses the recognition that memantine is useful to treat symptoms of Parkinson's disease, such as levodopa-induced dyskinesia (LID). Without wishing to be bound by any theory, an extended-release combination of acamprosate and memantine may be particularly useful for treating symptoms of Parkinson's disease, such as levodopa-induced dyskinesia (LID).

In some embodiments, memantine is in a dose of from about 5 mg to about 40 mg, such as about 5 mg, about 10 mg, about 15 mg, about 20 mg, about 25 mg, about 30 mg, about 35 mg, or about 40 mg, or any ranges between any two values. In some embodiments, the total daily dosage of memantine is from about 5 mg to about 40 mg, such as about 5 mg, about 10 mg, about 15 mg, about 20 mg, about 25 mg, about 30 mg, about 35 mg, or about 40 mg, or any ranges between any two values.

In some embodiments of a composition further comprising memantine, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 10 mg or about 15 mg memantine or about 1000 mg acamprosate and about 10 mg or about 15 mg memantine.

In some embodiments, a composition comprising acamprosate and memantine is for use in treating levodopa-induced dyskinesia in patients with Parkinson's disease. In some embodiments, a method of treating levodopa-induced dyskinesia in patients with Parkinson's disease comprises administering a therapeutically effective dose of a composition comprising acamprosate and memantine to a patient in need thereof.

In some embodiments, a provided composition further comprises molindone. Molindone (trade name Moban®) is an antipsychotic drug used to treat schizophrenia and psychosis. It is the only neuroleptic associated with weight loss rather than weight gain. Without wishing to be bound by any theory, an extended-release combination of acamprosate and molindone may be particularly useful for treating psychosis and obesity or metabolic syndrome, because acamprosate may reduce the risk that molindone will induce tardive dyskinesia (TD).

In some embodiments, molindone is in a dose of from about 5 mg to about 50 mg, such as about 5 mg, about 10 mg, about 20 mg, about 25 mg, about 30 mg, about 40 mg, or about 50 mg, or any ranges between any two values. In some embodiments, a total daily dosage of molindone is from about 10 mg to about 200 mg, such as about 10 mg, about 25 mg, about 50 mg, about 75 mg, about 100 mg, about 150 mg, or about 200 mg, or any ranges between any two values.

In some embodiments of a composition further comprising molindone, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 5 mg molindone, about 800 mg acamprosate and about 50 mg molindone, or about 1000 mg acamprosate and about 10 mg molindone.

In some embodiments, a composition comprising acamprosate and molindone is for use in treating psychosis and obesity or metabolic syndrome. In some embodiments, a method of treating psychosis and obesity or metabolic syndrome comprises administering a therapeutically effective dose of a composition comprising acamprosate and molindone to a patient in need thereof.

In some embodiments, a provided composition further comprises prazosin. Prazosin (trade names Minipress®, Vasoflex®, Pressin® or Hypovase®) is a sympatholytic drug used to treat high blood pressure and anxiety, PTSD, and panic disorder. It is an alpha-adrenergic blocker that is specific for the alpha-1 receptors. Without wishing to be bound by any theory, combining prazosin with an extended release formulation of acamprosate provided herein may enhance its central nervous system (CNS) levels relative to its systemic levels, as prazosin is both a substrate of and an inhibitor of the ABCG2 efflux pump, which determines the level of prazosin in the CNS. The enhanced CNS levels may reduce the hypotension seen early on in treatment with prazosin, mitigate prazosin food effect, and/or improve the efficacy of prazosin.

In some embodiments, prazosin is in a dose of from about 0.5 mg to about 15 mg, such as about 0.5 mg, about 1 mg, about 5 mg, about 10 mg, or about 15 mg, or any ranges between any two values. In some embodiments, a total daily dosage of prazosin is from about 1 mg to about 40 mg, such as about about 1 mg, about 5 mg, about 10 mg, about 15 mg, about 20 mg, about 30 mg, or about 40 mg, or any ranges between any two values.

In some embodiments of a composition further comprising prazosin, acamprosate is in a dose of from about 400 mg to about 1500 mg, such as about 400 mg, about 500 mg, about 800 mg, about 1000 mg, about 1300 mg, or about 1500 mg, or any ranges between any two values. In some such embodiments, a composition comprises about 800 mg acamprosate and about 1 mg prazosin or about 1000 mg acamprosate and about 10 mg prazosin.

In some embodiments, a composition comprising acamprosate and prazosin is for use in treating PTSD (e.g., PTSD with nightmares). In some embodiments, a method of treating PTSD (e.g., PTSD with nightmares) comprises administering a therapeutically effective dose of a composition comprising acamprosate and prazosin to a patient in need thereof.

### Uses of Provided Compositions

### Methods of Treatment

The present disclosure also provides methods of treating a disease, disorder, or condition, such as a neuropsychiatric disorder, in a patient in need thereof, such treatment comprising administering to said patient a composition or a unit dosage form of a composition described herein.

In some embodiments, a disease, disorder, or condition is selected from psychogenic nonepileptic seizures (PNES), tardive dyskinesia (TD), tardive akathisia, dystonia, blepharospasm, levodopa-induced dyskinesia (LID) in patients with Parkinson's disease, dyskinetic movements in Rett's Syndrome, dyskinetic movements in DiGeorge Syndrome, dyskinetic movements and dystonia in Wilson's disease, post-hypoxic myoclonus, simple tics, Tourette Syndrome (TS), obsessive-compulsive disorder (OCD), posttraumatic stress disorder (PTSD), symptoms of schizophrenia, depression, bipolar disorder, autism spectrum disorders, autistic symptoms in Fragile X syndrome, alcoholism, tinnitus, generalized anxiety disorder, and repetitive and stereotypic self-injurious behaviors (SIB) in persons with developmental disabilities such as biting, skin-picking, hitting oneself, and head-banging.

In some embodiments, provided methods comprise reducing anxiety and/or agitation in a patient receiving a neuroleptic, anti-anxiety, or antidepressant medication.

In some embodiments, provided methods comprise reducing severity of or reducing or delaying the onset of a disease, disorder, syndrome condition. In some embodiments, provided methods comprise treating or selecting a particular patient, group or population of patients to receive treatment.

In some embodiments, provided methods comprise treating or selecting a patient in need of taking acamprosate or another medication described herein with food, without food, in a fed or in a fasted stated. In some embodiments, a patient is in a fed state. In some embodiments, a patient is in a fasted state. In some embodiments, a patient, group or population is susceptible to GI side effects of acamprosate and/or another medication, and needs to take the acamprosate and/or the another medication with or without food, and therefore desires to take the acamprosate and the another medication in the same manner.

In some embodiments, a patient, group or population is one that needs to take about 1000 mg, about 1500 mg, about 2000 mg, about 2500 mg, about 3000 mg, about 3500 mg or about 4000 mg of acamprosate daily.

In some cases, a patient, group or population is one susceptible to non-compliance with a treatment regimen comprising acamprosate and/or another medication, or one that needs a treatment regimen with fewer side effects in order to encourage or facilitate compliance. In some embodiments, a patient, group or population is one that needs to avoid or delay the onset of side effects of a neuroleptic treatment, such as the onset of TD. In some embodiments, a patient, group, or population is one that experiences anxiety or depression, and/or is taking a medication for anxiety or depression and needs to treat a condition.

In some embodiments, a patient, group, or population comprises pediatric patients. In some embodiments, a patient, group, or population comprises adolescent patients. In some embodiments, a patient, group, or population comprises elderly patients. In some embodiments, a patient, group, or population comprises patients that experience difficulty swallowing a pill (e.g., pediatric, adolescent, or elderly populations in need of treatment with acamprosate).

In some embodiments, a provided composition is administered once, twice or three times daily. In some embodiments, provided methods comprise, for example, administering to a patient in need thereof a total daily dosage of acamprosate of from about 500 mg to about 4000 mg per day. In some embodiments, a method comprises, for example, administering to a patient acamprosate at a daily dosage of about 1000 mg to about 2000 mg, about 2000 mg to about 3000 mg, or about 3000 mg to about 4000 mg, on a once-a-day schedule with or without food (i.e., in a fed or a fasted state). In some embodiments, a method comprises administering to a patient acamprosate at a daily dosage of 1000 mg to 4000 mg once a day with or without food (i.e., in a fed or a fasted state).

In some embodiments, a method comprises administering to a patient acamprosate at a daily dosage of about 1000 mg to about 2000 mg, about 2000 mg to about 3000 mg, or about 3000 mg to about 4000 mg, on a twice-a-day schedule with or without food (i.e., in a fed or a fasted state). In some embodiments, a method comprises administering to a patient acamprosate at a daily dosage of about 1000 mg to about 2000 mg, about 2000 mg to about 3000 mg, or about 3000 mg to about 4000 mg, on a thrice-a-day schedule with or without food (i.e., in a fed or a fasted state).

In some embodiments, provided methods comprise administering to a patient about 4000 mg of acamprosate per day. In some embodiments, provided method comprise administering to a patient about 3200 mg of acamprosate per day.

In some embodiments, provided methods comprise administering to a patient acamprosate in two tablet compositions twice a day wherein each tablet composition comprises about 800 mg to about 1000 mg of acamprosate (e.g., so that the patient is administered about 3200 mg to about 4000 mg of acamprosate per day). In some embodiments, once or twice daily acamprosate administration is at a dosage of about 1000 mg, less than or equal to 4000 mg, more than 1000 mg, or equal to or less than 3000 mg, (e.g., in a dosage of about 1000 mg to about 2000 mg, or about 2000 mg to about 3000 mg, or about 3000 mg to about 4000 mg).

Without being limited thereto, in some embodiments, acamprosate can be administered as one, two, three, or four units of a dosage form, for example, as one, two, three, or four tablet compositions. In some embodiments, a unit dosage form has a total weight of up to 1500 mg, (e.g., from about 1000 mg to about 1500 mg, from about 1100 mg to about 1300 mg, or from about 1100 mg to about 1200 mg). In some embodiments, a unit dosage form weight can be between about 400 mg and about 1500 mg, between about 800 mg and about 1200 mg, between about 1000 mg and about 1200 mg, between about 1100 mg and about 1200 mg, or any value or sub range within those ranges.

In some embodiments, provided methods comprises administering a composition comprising acamprosate to a patient in need thereof, wherein the patient is in a fed or fasted state, and the patient may choose to take each individual dose on each individual occasion in either the fed state or the fasting state. In some embodiments, a patient is administered a composition or a unit dose of a composition described herein in a fasted state. In some embodiments, a patient is administered a composition or a unit dose of a composition described herein in a fed state. In some embodiments, provided methods specifically exclude administration in the fasted or the fed state.

In some embodiments, a patient is administered a composition or a unit dose of a composition described herein immediately prior to food intake (e.g., within 30 minutes or within 60 minutes prior to taking food), with food, or soon after food intake (e.g., within 30 minutes, within 60 minutes or within 2 hours after food intake). In some embodiments, a patient is administered a composition or a unit dose of a composition described herein without food, for example, after an overnight fast, or not less than 30-60 minutes prior to a meal or not less than 1 hour, 2 hours, 3 hours after a meal, or more. In some embodiments, a patient is administered a composition or a unit dose of a composition described herein at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours or more after food intake, or any time there between. In some embodiments, a patient is administered a composition or a unit dose of a composition described herein at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours or more before food intake, or any time there between.

Without wishing to be bound by theory, due to fed/fasted equivalence of provided compositions, provided compositions provide flexibility to a patient with respect to when and/or how a provided composition is taken, which favors patient compliance. For example, in some embodiments, when a composition is given TID (e.g, three times a day), it can be taken, for example, with food for one or two doses and without food for one or two doses. In some embodiments, when a patient is already taking other medications, doses of the acamprosate compositions provided herein can be taken at the same time as the other medications. Most patients with neuropsychiatric disorders will be on other medications and may tolerate the other medication better if taken with or without food. Accordingly, in some embodiments, provided compositions are administered to a patient in need thereof in any suitable manner (e.g., with or without food).

In some embodiments, acamprosate is administered once or twice daily in order to achieve a particular total daily dosage, and a composition comprising acamprosate is formulated to release at least 50% of the acamprosate over a 2-8 hour period or any sub value or sub range there between. In some embodiments, at least 50% of the acamprosate is released within the first 4 hours. In some embodiments, at least 90% of the acamprosate is released from the composition within 8 hours.

### Combination Therapy

The present disclosure also provides use of provided compositions in combination with one or more additional therapeutic agents. In some embodiments, methods described herein further comprise administering provided compositions of acamprosate with one or more additional therapeutic agents, such as first-generation neuroleptic (antipsychotic) drugs, second generation neuroleptic drugs, selective serotonin reuptake inhibitors (SSRIs), serotonin norepinephrine reuptake inhibitors (SNRIs), anti-nausea drugs, NMDA modulators, and/or an α_{2A} adrenergic receptor agonists, or any combination thereof. In some embodiments, an additional therapeutic agent is administered with acamprosate in a fixed dose form or in separate dosage forms simultaneously or sequentially, such as at a different time, and/or on the same or different dosing schedule. As noted above, combination therapy (e.g., administering acamprosate in combination with one or more additional therapeutic agents) can provide benefits, such as increased patient compliance, increased therapeutic effects, and reduced side effects.

In some embodiments, provided compositions comprise acamprosate in a dose of from about 500 mg to about 4000 mg or from about 800 mg to about 1200 mg, and an additional therapeutic agent in a dose ranging from half of the lower end of its usual dosage range to the upper end of its dosage range. In some embodiments, a total daily dosage of acamprosate is from about 500 mg to about 4000 mg, or from about 1000 mg to about 4000 mg, or from about 1600 mg to about 4000 mg.

In some embodiments, provided methods comprise administering a fixed dose combination, comprising acamprosate and an additional therapeutic agent, to a patient in need thereof. In some embodiments, a fixed dose combination is used for treating any of the disorders treated with, for example, neuroleptic drugs, including schizophrenia, schizoaffective disorder, bipolar disorder, major depression, delusional disorder, organic psychoses, delirious agitation, or nausea and vomiting. In some such embodiments, a fixed dose combination is administered once-daily or twice-daily, typically with a single tablet composition given each time.

Without wishing to be bound by theory, for a given dosage of neuroleptic, a fixed dose combination as described herein can give equal or greater benefit for the neuropsychiatric disorder or symptoms being treated, compared to administration of the neuroleptic alone. Compared with the same dose of a first-generation neuroleptic given without acamprosate, fixed dose combinations described herein entail a lower risk of tardive dyskinesia and other tardive movement disorders, and result in movement disorder of lesser severity, if they cause one at all. In contrast with second-generation neuroleptics of equal therapeutic efficacy, a combination comprising a first generation neuroleptic and acamprosate may carry less risk of significant metabolic disturbances including weight gain, glucose intolerance, and increased risk of atherosclerotic cardiovascular disease.

The present disclosure also provides methods of reducing risk or delaying onset of tardive dyskinesia, the method comprising administering to a patient in need thereof a combination as described herein. In some embodiments, the method comprises administering to a patient in need thereof a composition comprising acamprosate and a neuroleptic medication.

In some embodiments, a method comprises administering to a patient in need thereof a composition comprising acamprosate and a first generation neuroleptic medication. At present the first-generation, high-potency neuroleptic medications are avoided because they are more likely than second-generation neuroleptic drugs to produce tardive dyskinesia. Nevertheless, first-generation neuroleptic medications are generally no less efficacious in treating psychosis than second-generation neuroleptic medications. In addition, second-generation neuroleptic medications usually are more expensive and have serious metabolic side effects with potentially life-threatening consequences. Accordingly, in some embodiments, a patient has tardive dyskinesia and requires continued neuroleptic therapy for a chronic mental disorder. In some embodiments, a patient has tardive dyskinesia and requires continued neuroleptic therapy with a first-generation neuroleptic medication for a chronic mental disorder.

In some embodiments, a method comprises administering to a patient in need thereof a composition comprising acamprosate and a second generation neuroleptic medication. Second-generation neuroleptic medications still carry some risk of TD. Accordingly, in some embodiments, a patient has tardive dyskinesia and requires continued neuroleptic therapy with a second-generation neuroleptic medication for a chronic mental disorder.

### EXAMPLES

### Example 1: Prior Fogel Formulations with Carbomer Homopolymer Type B

The present Example describes certain exemplary formulations described in the Prior Fogel Filings that utilize a carbomer homopolymer type B (specifically, Carbopol® 974) and achieve desirable manufacturability and delivery characteristics for a therapeutic agent (e.g., an acamprosate agent). These exemplary formulations were manufactured as 400 mg (Formulation 1) and 800 mg (Formulation 2) tablets. In some embodiments, these tablets were standard round bi-convex white tablets with beveled edges. Both tablet strengths were spray coated with Opadry® II White (Colorcon, Inc.) for ease of swallowing. Purified water is the vehicle for the Opadry; it evaporates during the coating process. The total weight of the coating was between 2% and 4% of the pre-coating weight.

The tablets prior to coating comprised the ingredients in the following table:

TABLE 1

| | | Formu- | Formu- |
|---|---|---|---|
| | | lation 1 | lation 2 |
| | | Amount | Amount |
| Ingredient | Function | (mg) | (mg) |
| Acamprosate calcium | Active ingredient | 400 | 800 |
| Povidone K-90 | Binder | 50 | 50 |
| Microcrystalline cellulose | Diluent | 320 | 100 |
| Colloidal silicon dioxide | Glidant | 10 | 10 |
| Citric acid | Acidulant | 60 | 0 |
| Carbopol ® 974P | Polymer | 60 | 60 |
| Carboxymethyl cellulose | Polymer | 40 | 40 |
| Starcap 1500 ® | Disintegrant | 40 | 40 |
| Talc powder | Filler | 10 | 10 |
| Magnesium stearate | Lubricant | 10 | 10 |

| | | |
|---|---|---|
| Total prior to coating | 1000 | 1120 |

Dissolution profiles of these 400 mg and 800 mg acamprosate tablets were determined, and were reported in certain of the Prior Fogel Filings. Specifically, 400 mg or 800 mg tablets were dissolved in either acetate solution (pH 4.5) or 1N HCl (pH 1.0). The percentage of the active ingredient released into the solution was determined at 1, 2, 4, 6, 10, and 12 hours. Each release profile was assessed in six different test vessels. The following Tables 2-5 display the results, demonstrating that release is approximately linear with the square root of time, and furthermore demonstrating that the release is comparable across the pH range tested (i.e., pH 1.0 to pH 4.5). The fourth column in each Table displays the amount of drug that would be released if the release were exactly proportional to the square root of time, with a specified coefficient that ranges from 0.27 to 0.3.

TABLE 2
Release of Acamprosate
from Formulation 1 in Acetate Solution (pH 4.5) (n = 6)

| | | Mean % | | S.D. of % | | Maxi- |
|---|---|---|---|---|---|---|
| | | of Total | 27% * | of Total | Minimum | mum |
| Time | SQRT | Drug | SQRT | Drug | % | % |
| (hours) | Time | Released | Time | Released | Released | Released |
| 0 | 0.0000 | 0.00 | 0.00 | 0.00 | 0 | 0 |
| 1 | 1.0000 | 24.09 | 27.00 | 1.40 | 23 | 27 |
| 2 | 1.4142 | 36.09 | 38.18 | 2.34 | 34 | 40 |
| 4 | 2.0000 | 54.39 | 54.00 | 4.02 | 49 | 60 |
| 6 | 2.4495 | 70.11 | 66.14 | 4.15 | 64 | 76 |
| 10 | 3.1623 | 87.67 | 85.38 | 4.21 | 83 | 95 |
| 12 | 3.4641 | 92.31 | 93.53 | 4.38 | 87 | 99 |

TABLE 3
Release of Acamprosate
from Formulation 1 in 0.1N HCl (pH 1.0) (n = 6)

| | | Mean % | | S.D. of % | | Maxi- |
|---|---|---|---|---|---|---|
| | | of Total | 27% * | of Total | Minimum | mum |
| Time | SQRT | Drug | SQRT | Drug | % | % |
| (hours) | Time | Released | Time | Released | Released | Released |
| 0 | 0.0000 | 0.00 | 0.00 | 0.00 | 0 | 0 |
| 1 | 1.0000 | 31.60 | 27.00 | 1.58 | 24 | 27 |
| 2 | 1.4142 | 44.83 | 38.18 | 3.20 | 36 | 41 |
| 4 | 2.0000 | 63.20 | 54.00 | 4.42 | 56 | 62 |
| 6 | 2.4495 | 75.27 | 66.14 | 5.42 | 69 | 74 |
| 10 | 3.1623 | 91.31 | 85.38 | 3.59 | 85 | 91 |
| 12 | 3.4641 | 95.99 | 93.53 | 2.96 | 90 | 96 |

TABLE 4
Release of Acamprosate from
Formulation 2 in Acetate Solution (pH 4.5) (n = 6)

| | | Mean % | | S.D. of % | | Maxi- |
|---|---|---|---|---|---|---|
| | | of Total | 30% * | of Total | Minimum | mum |
| Time | SQRT | Drug | SQRT | Drug | % | % |
| (hours) | Time | Released | Time | Released | Released | Released |
| 0 | 0.0000 | 0.00 | 0.00 | 0.00 | 0 | 0 |
| 1 | 1.0000 | 31.60 | 30.00 | 1.58 | 29 | 34 |
| 2 | 1.4142 | 44.83 | 42.43 | 3.20 | 42 | 51 |
| 4 | 2.0000 | 63.20 | 60.00 | 4.42 | 58 | 64 |
| 6 | 2.4495 | 75.27 | 73.48 | 5.42 | 70 | 84 |
| 10 | 3.1623 | 91.31 | 94.87 | 3.59 | 87 | 97 |
| 12 | 3.4641 | 95.99 | 100.00 | 2.96 | 91 | 100 |

TABLE 5
Release of Acamprosate
from Formulation 2 in 0.1N HC1 (pH 1.0) (n = 6)

| | | Mean % | | S.D. of % | | Maxi- |
|---|---|---|---|---|---|---|
| | | of Total | 29% * | of Total | Minimum | mum |
| Time | SQRT | Drug | SQRT | Drug | % | % |
| (hours) | Time | Released | Time | Released | Released | Released |
| 0 | 0.0000 | 0.00 | 0.00 | 0.00 | 0 | 0 |
| 1 | 1.0000 | 28.11 | 29.00 | 1.21 | 26 | 29 |
| 2 | 1.4142 | 41.56 | 41.01 | 1.77 | 40 | 44 |
| 4 | 2.0000 | 61.48 | 58.00 | 2.38 | 57 | 63 |
| 6 | 2.4495 | 75.41 | 71.04 | 1.61 | 73 | 77 |
| 10 | 3.1623 | 92.24 | 91.71 | 0.55 | 92 | 93 |
| 12 | 3.4641 | 96.62 | 100.00 | 0.65 | 96 | 97 |

Table 6 describes % swelling and firmness characteristics of Formulation 2, evaluated as described in Example 5.

TABLE 6
% Swelling and firmness of tablets of Formulation 2

| Property | pH 1.0 | pH 4.5 | pH 6.8 |
|---|---|---|---|
| % Swelling (length) | 127% | 119% | 110% |
| % Swelling (width) | 158% | 147% | 150% |
| Firmness | 4.5 | 3 | 4 |

### Example 2: Prior Fogel Formulations with Carbomer Homopolymer Type A

The present Example describes certain exemplary formulations described in the Prior Fogel Filings that utilize a carbomer homopolymer type A (specifically, Carbopol® 971) and achieve desirable manufacturability and delivery characteristics for a therapeutic agent (e.g., an acamprosate agent). In particular, Table 7 presents the ingredients included in tablets comprising various Prior Fogel Formulations.

TABLE 7

| | | Amount (mg) | | |
|---|---|---|---|---|
| | | Formu- | Formu- | Formu- |
| | | lation | lation | lation |
| Component | Function | 2 | 3 | 4 |
| Acamprosate | Active ingredient | 800 | 800 | 800 |
| Microcrystalline | Diluent | 100 | — | — |
| Cellulose | | | | |
| Carbopol ® 971P | Polymer | — | 60 | 80 |
| (Type A) | | | | |
| Carbopol ® 974P | Polymer | 60 | — | — |
| (Type B) | | | | |
| Povidone K-90 | Binder | 50 | 50 | 50 |
| CMC 7HF | Polymer | 40 | 40 | 20 |
| Starcap 1500 ® | Disintegrant | 40 | 40 | 40 |
| Colloidal Silicon | Glidant | 10 | — | — |
| Dioxide | | | | |
| Talc Powder | Filler | 10 | 10 | 10 |
| Magnesium stearate | Lubricant | 10 | 10 | 10 |
| Avicel ® PH102 | Binder | — | 100 | 100 |
| Cabosil | Glidant | — | 10 | 10 |

| | | | |
|---|---|---|---|
| Total prior to coating | 1120 | 1120 | 1120 |

Table 8 displays the results of contacting the tablets described in Example 2 with either acetate solution (pH 4.5) or 0.1 N HCl (pH 1.0). In particular, (i) the swelling results are similar to those of Formulation 2 in Example 1; (ii) no erosion was observed; (iii) the % swelling is greater in Formulation 4 than in Formulation 3; and (iv) the swollen tablets are intact and firm for 6 hours during the swelling test. The results in Table 9 show that the dissolution profile is substantially proportional to the square root of time and is equivalent to the reference 800 mg tablets (Formulation 2) of Example 1.

TABLE 8
Swell test of 800 mg tablets of Formulations 3 and 4

| | Formulation 3 | | Formulation 4 | |
|---|---|---|---|---|
| Property | HCl | Acetate | HCl | Acetate |
| Initial Length (cm) | 1.28 | 1.28 | 1.28 | 1.28 |
| Length 5 min (cm) | 1.4 | 1.4 | 1.45 | 1.45 |
| Length 30 min (cm) | 1.5 | 1.5 | 1.55 | 1.5 |
| Length 1 hr (cm) | 1.55 | 1.52 | 1.6 | 1.52 |
| Length 2 hr (cm) | 1.65 | 1.55 | 1.7 | 1.55 |
| Length 6 hr (cm) | 1.9 | 1.65 | 1.95 | 1.7 |
| % Swelling (length) | 148% | 129% | 152% | 133% |
| Firmness Rating | 4.5 | 4 | 4.8 | 4 |
| Initial Thickness (cm) | 0.77 | 0.78 | 0.78 | 0.78 |
| Final Thickness (cm) | 1.5 | 1.3 | 1.55 | 1.3 |
| % Swelling (width) | 195% | 167% | 199% | 167% |

### Firmness Rating System:

1. Shapeless after 6 hours in acid
2. Loosely retains shape, very soft
3. Retains shape, but offers no resistance
4. Can be picked up, slight resistance
5. Very firm and elastic

TABLE 9
Release Profiles at pH 1.0 and pH 4.5

| | | Formu- | Formu- | Formu- |
|---|---|---|---|---|
| | | lation | lation | lation |
| pH | Release Profile | 2 | 3 | 4 |
| 1.0 | % Dissolution (t = 1 h) | 29.6 | 30.21 | 29.27 |
| | % Dissolution (t = 2 h) | 42.1 | 42.22 | 44.21 |
| | % Dissolution (t = 4 h) | 58.9 | 58.63 | 62.71 |
| | % Dissolution (t = 6 h) | 71.3 | 70.15 | 74.75 |
| | % Dissolution (t = 10 h) | 87 | 84.94 | 89.04 |
| | % Dissolution (t = 12 h) | 91 | 88.14 | 92.79 |
| | f2 | | 86.8 | 78.28 |
| 4.5 | % Dissolution (t = 1 h) | 30.9 | 36.41 | 34.24 |
| | % Dissolution (t = 2 h) | 44.3 | 49.75 | 47.42 |
| | % Dissolution (t = 4 h) | 61.6 | 68.76 | 64.53 |
| | % Dissolution (t = 6 h) | 74.4 | 80.72 | 77.94 |
| | % Dissolution (t = 10 h) | 89 | 93.94 | 92.93 |
| | % Dissolution (t = 12 h) | 92 | 96.34 | 95.68 |
| | f2 | | 61.9 | 72.29 |

### Example 3: Preparation of Tablet Compositions

In work documented in the present Examples, tablet compositions prior to coating were prepared according to the following general two-step process:
1. Wet granulation step: Materials (“granular materials”) were weighed, sieved, and blended. Then, granulation was effected using purified water as the granulating agent to form wet granules. The wet granules were sieved and dried until the % LOD (loss on drying) was no more than 2%, resulting in the dry granules.
2. Extra-granulation step: The dry granules were sieved and blended with the additional materials (“extra-granular materials”). The final blend of the granules and extra-granular materials was compressed into tablets. In some cases, oval punches 0.758″×0.350″ embossed with “BK” on one side were used for compression.

The compressed tablets were evaluated for various parameters, including target weight, hardness, thickness, and friability.

In some aspects, the process further comprised a coating step. The coating step was performed on the compressed tablets. Various coating parameters for a pan coater (pan size: 12 inch) were used for coating:
(1) Coating system: 20% Opadry® II white
(2) Spray rate: 8 gm/min
(3) Pan speed: 18 rpm
(4) Pan load: 900 g
(5) Air flow rate: 200 CFM
(6) Pan differential pressure: −0.1 psi
(7) Atomic air pressure: 30 psi
(8) Pattern air pressure: 20 psi
(9) Setting temperature: 48-58° C.; Inlet temperature: 44.0-63.0° C.; Exhaust temperature: 39.0-49.0° C.; and
(10) Target weight gain: 2-4%.

### Example 4: Preparation of Tablet Compositions as Single Oval Tablets

The process of Example 3 was used for preparing the Prior Fogel Formulations (Formulations 1 and 2 of Example 1 and Formulations 3 and 4 of Example 2). The tablets were standard round bi-convex white tablets with beveled edges. While this process can usefully provide desirable formulations for the preparation of round tablets, the present disclosure identifies a source of variability with the process when the process is used to prepare oval tablets. As disclosed above, oval tablets are desirable for a number of reasons, including ease of swallowing and increased patient compliance.

Attempts to make oval tablets using equivalent manufacturing process parameters for the preparation of Prior Fogel Formulations did not result in oval tablets with acceptable physical properties, i.e., the resulting oval tablets did not possess acceptable hardness (<2-3 kp) or friability (>3%).

The source of the problem was traced to the wet granulation step. In particular, granulation with purified water provided a wet powder mass that did not bind well and, as a result, led to overwetting of the material and routine clogging of the screen during sieving. Various modifications of the process parameters (e.g., reducing the speed of the granulator impeller and chopper, reducing the rate of water addition, etc.) did not lead to acceptable tablets.

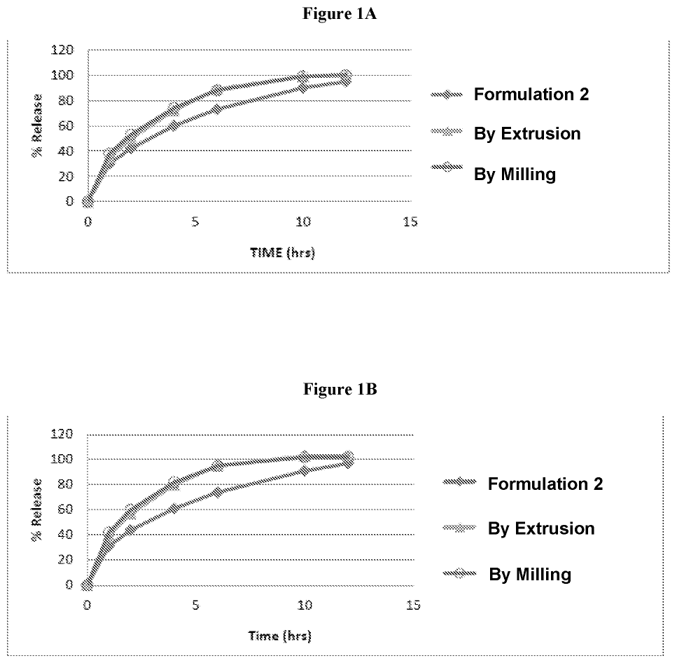

Two alternative methods were then used to prepare granules: (i) extrusion or (ii) milling (e.g., with a Fitzmill) without a screen. Oval tablets prepared using either (i) extrusion or (ii) milling (e.g., with a Fitzmill) without a screen provided tablets with a hardness in an acceptable range of 15-18 kp and friability <1.0%. However, dissolution profiles of the resulting oval tablets from either of these processes indicated that, although physical properties comparable to the round tablets were achieved, the dissolution profile was somewhat faster (6-8 hours for 100% active agent release) with the oval tablets compared to the round tablets (8-10 hours for 100% active agent release) at both pH 1.0 (FIG. 1A) and pH 4.5 (FIG. 1B).

In addition to the use of (i) extrusion or (ii) milling (e.g., with a Fitzmill) without a screen, further modifications to the process were evaluated:
(a) Addition of 40 mg carboxymethyl cellulose (CMC 7HF) to the granular materials instead of to the extra-granular materials resulted in a formulation that compressed well and provided tablets with acceptable physical properties, but still exhibited a faster release profile than that of Formulation 2.
(b) Addition of 40 mg carboxymethyl cellulose (CMC 7HF) and 60 mg carbomer homopolymer type B to the granular materials instead of to the extra-granular materials resulted in a formulation that compressed well and provided tablets with acceptable physical properties.
(c) Addition of 60 mg carbomer homopolymer type B to the granular materials instead of the extra-granular materials resulted in a formulation that did not compress well and provided tablets with poor physical properties.
(d) Addition of an additional 40 mg carboxymethyl cellulose (CMC 7HF) to the formulation used to prepare Formulation 2, such that 40 mg CMC 7HF remained in the extra-granular materials and an additional 40 mg CMC 7HF was added to the granular materials, resulted in an oval tablet with acceptable physical properties and a release profile that closely matched that of Formulation 2.

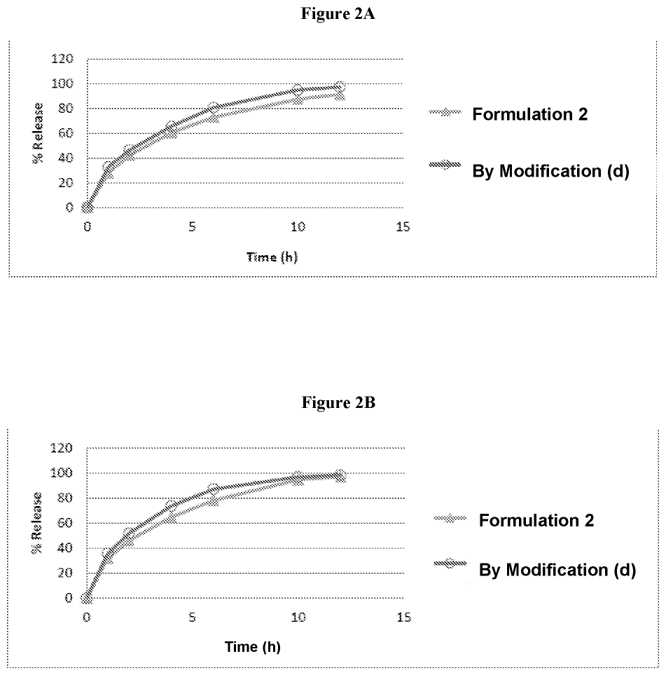

The tablet resulting from modification (d) has an increased tablet weight of 1,160 mg absent any coating, as compared to 1,120 mg absent any coating for tablets of Formulation 2. Oval tablets prepared using modification (d) exhibited dissolution profiles at pH 1.0 (FIG. 2A) and pH 4.5 (FIG. 2B) that closely match those of Formulation 2.

Table 10 presents Formulation 5, the oval tablets prepared according to modification (d), described above.

TABLE 10
Formulation 5

| | | | Amount |
|---|---|---|---|
| | Component | Function | (mg) |
| Granular | Acamprosate | Active | 800 |
| Materials | Calcium | ingredient | |
| (mg) | Avicel ® Ph 102 | | |
| | (Microcrystalline | Diluent | 100 |
| | cellulose) | | |
| | Povidone K90 | Binder | 50 |
| | CMC 7HF | Polymer | 40 |
| | Colloidal Silicon | Glidant | 10 |
| | Dioxide | | |
| Extra- | Carbopol ® 974 P | Polymer | 60 |
| Granular | CMC 7HF | Polymer | 40 |
| Materials | Starcap 1500 | Disintegrant | 40 |
| (mg) | Talc powder | Filler | 10 |
| | Magnesium Stearate | Lubricant | 10 |

| | |
|---|---|
| Total prior to coating | 1160 |
| Tablet Shape | oval |

Among other things, the present disclosure provides a solution to the above-identified problem and, furthermore, provides methodologies for developing and/or preparing formulations with acceptable physical properties and release profiles. For example, in some embodiments, the present disclosure describes a process in which granules are prepared and then combined with extra-granular materials before compression into tablets. In some embodiments, the present disclosure describes a process for formulating the Prior Fogel Formulations as oval tablets. In some embodiments, a process for formulating the Prior Fogel Formulations as oval tablets comprises the addition of 40 mg CMC 7HF to the granular materials, according to modification (d).

### Example 5: Testing of Formulations

The Examples herein describe formulations of the present disclosure and demonstrate the physical characteristics of the tablets produced using such formulations. Characteristics assessed include, for example, dissolution profiles, erosion properties of the tablets, and swelling properties of the tablets.

The dissolution profiles of prepared formulations were compared to the reference product 800 mg tablets of Example 1 (Formulation 2). The prepared formulations were tested at pH 1.0 (0.1 N HCl), pH 4.5 (acetate buffer), and pH 6.8 (phosphate buffer) for up to 12 hrs. The f2 similarity factor was calculated using the following equation to determine the equivalent performance with the reference product Formulation 2:

f2=50·log{[I+(1/n)l:t=1n(Rt−Tt)2]−0.5·100}

The similarity factor is a logarithmic reciprocal square root transformation of the sum of squared error and is a measurement of the similarity in the percent (%) of dissolution between the two curves, where n is the number of time points, Rt is the dissolution value of the reference batch at time t, and Tt is the dissolution value of the test batch at time t.

The erosion properties of prepared formulations were assessed (n=4) as follows: initial weights of the individual tablets (Wi) were recorded and erosion studies were performed at pH 1.0 (0.1 N HCl), pH 4.5 (acetate buffer), and pH 6.8 (phosphate buffer) for 2 hr and/or 6 hr. Tablets were carefully removed from the medium and dried at 55° C. overnight. The individual dried final weights were recorded (Wt). The % of the material eroded is calculated by the following formula:

% Erosion=(Wf−Wi)/Wi*100%

The swelling properties of prepared formulations were assessed (n=3) as follows: the initial length and thickness were recorded and the swelling test was performed at pH 1.0 (0.1 N HCl), pH 4.5 (acetate buffer), and pH 6.8 (phosphate buffer) for up to 6 hr. % Swelling of the length and thickness were recorded. Firmness was determined on a 1-5 scale, as described in Example 2.

The results of the above assessments are included and described in the following examples which detail provided formulations with carbomer homopolymers.

### Example 6: Formulations with Carbomer Homopolymer Type B

This Example summarizes exemplary useful compositions provided by the present disclosure that meet certain performance requirements as described herein.

In particular, Table 11 presents the ingredients included in various provided formulations, prepared according to a two-step procedure as described above in Example 3. Release profiles for 800 mg tablets prepared from Formulations 6-8 were obtained and the data are presented in Tables 11-15. The data were consistent with the release data for the reference Formulation 2 at the three pH values tested. Additionally, the 800 mg tablets prepared from Formulations 6-8 demonstrated suitable swelling properties and firmness as is shown in Table 15.

TABLE 11

| | | | Amount (mg) | | |
|---|---|---|---|---|---|
| | | | Formulation | Formulation | Formulation |
| | Component | Function | 6 | 7 | 8 |
| Granular | Acamprosate | Active | 800 | 800 | 800 |
| Materials | Calcium | ingredient | | | |
| (mg) | Avicel ® Ph 102 | — | 100 | — | |
| | (Microcrystalline | Diluent | | | |
| | cellulose) | | | | |
| | Povidone K90 | Binder | 50 | 40 | 40 |
| | EUDRAGIT ® | Polymer | — | 10 | 10 |
| | FS 30D* | | | | |
| | Sugar Spheres | Carrier | 100 | — | 100 |
| | Colloidal Silicon | Glidant | 10 | 10 | 10 |
| | Dioxide | | | | |
| Extra- | Carbopol ® 974P | Polymer | 90 | 90 | 90 |
| Granular | CMC 7HF | Polymer | 10 | — | — |
| Materials | Starcap 1500 | Disintegrant | — | 10 | 10 |
| (mg) | Gelatin Type B | Polymer | 40 | 40 | 40 |
| | Talc powder | Filler | 10 | 10 | 10 |
| | Magnesium Stearate | Lubricant | 10 | 10 | 10 |

| | | | |
|---|---|---|---|
| Total prior to coating (mg) | 1120 | 1120 | 1120 |
| Tablet Shape | oval | oval | oval |

TABLE 12
Release profiles of tablets in 0.1N HCl (pH 1.0)

| | Formu- | Formu- | Formu- |
|---|---|---|---|
| Release Profile (pH 1.0) | lation 6 | lation 7 | lation 8 |
| % Erosion (t = 2 h) | 26.25 | 29.88 | 28.76 |
| % Dissolution (t = 1 h) | 28.47 | 31.12 | 28.78 |
| % Dissolution (t = 2 h) | 42.02 | 44.06 | 40.51 |
| % Dissolution (t = 4 h) | 61 | 62.46 | 57.94 |
| % Dissolution (t = 6 h) | 77.24 | 78.25 | 77.41 |
| % Dissolution (t = 10 h) | 92.3 | 93.85 | 90.95 |
| % Dissolution (t = 12 h) | 95.12 | 98.33 | 95.75 |
| f2 | 71.59 | 64.4 | 72.4 |
| Y-intercept, SQRT | 1.5699 | 2.4211 | 0.9569 |
| Slope, SQRT | 28.551 | 28.993 | 28.513 |
| R\hat{ }2, SQRT | 0.9902 | 0.9929 | 0.9917 |

TABLE 13
Release profiles of tablets in acetate solution (pH 4.5)

| | Formu- | Formu- | Formu- |
|---|---|---|---|
| Release Profile (pH 4.5) | lation 6 | lation 7 | lation 8 |
| % Erosion (t = 2 h) | 27.21 | 43.44 | 28.42 |
| % Dissolution (t = 1 h) | 34.98 | 38.05 | 36.44 |
| % Dissolution (t = 2 h) | 51.44 | 52.42 | 51.93 |
| % Dissolution (t = 4 h) | 73.55 | 74.03 | 73.16 |
| % Dissolution (t = 6 h) | 88.39 | 87.87 | 90.32 |
| % Dissolution (t = 10 h) | 95.36 | 96.5 | 96.6 |
| % Dissolution (t = 12 h) | 96.71 | 98.1 | 98.63 |
| f2 | 52.88 | 51.25 | 50.64 |
| Y-intercept, SQRT | 7.4475 | 8.3906 | 7.5718 |
| Slope, SQRT | 28.784 | 28.779 | 29.212 |
| R\hat{ }2, SQRT | 0.9529 | 0.9568 | 0.9556 |

TABLE 14
Release profiles of tablets in phosphate solution (pH 6.8)

| | Formu- | Formu- | Formu- |
|---|---|---|---|
| Release Profile (pH 6.8) | lation 6 | lation 7 | lation 8 |
| % erosion (t = 2 h) | 20.67 | 34.52 | 20.75 |
| % Dissolution (t = 1 h) | 27.3 | 28 | 28.47 |
| % Dissolution (t = 2 h) | 35.59 | 37.23 | 37.85 |
| % Dissolution (t = 4 h) | 41.68 | 44.75 | 45.49 |
| % Dissolution (t = 6 h) | 46.93 | 51.57 | 52.43 |
| % Dissolution (t = 10 h) | 54.6 | 59.62 | 60.61 |
| % Dissolution (t = 12 h) | 58.54 | 65.6 | 66.7 |
| f2 | 75.43 | 83.77 | 77.6 |
| Y-intercept, SQRT | 7.5381 | 6.8017 | 6.9146 |
| Slope, SQRT | 15.706 | 17.728 | 18.024 |
| R\hat{ }2, SQRT | 0.9431 | 0.9618 | 0.9618 |

TABLE 15
% Swelling and firmness of tablets

| Property | Conditions | Formulation 6 | Formulation 7 | Formulation 8 |
|---|---|---|---|---|
| % | 0.1N HCl, pH 1.0 | 127% | 130% | 127% |
| Swelling | Acetate Buffer, pH 4.5 | 112% | 115% | 117% |
| (length) | Phosphate Buffer, pH 6.8 | 115% | 116% | 121% |
| % | 0.1N HCl, pH 1.0 | 167% | 166% | 165% |
| Swelling | Acetate Buffer, pH 4.5 | 140% | 154% | 138% |
| (thickness) | Phosphate Buffer, pH 6.8 | 140% | 152% | 150% |
| Firmness | 0.1N HCl | 4.50 | 4.80 | 4.50 |
| | Acetate Buffer, pH 4.5 | 3.80 | 3.80 | 3.80 |
| | Phosphate Buffer, pH 6.8 | 4.20 | 4.20 | 4.20 |

### Example 7: Formulations with Carbomer Homopolymer Type C

This Example summarizes exemplary useful compositions provided by the present disclosure that meet certain performance requirements as described herein.

In particular, Table 16 presents the ingredients included in various provided formulations, prepared according to a two-step procedure as described above in Example 3. As can be seen in the release profile data in Table 17, Formulations 9-11 have release data that were equivalent to the reference Formulation 2. Formulation 12, which uses 140 mg carbomer homopolymer type C had a slower release rate as compared to the reference. Further release profile data were obtained for Formulation 9 at pH 4.5 and 6.8; these data were also equivalent to the reference Formulation 2, as shown in Tables 18 and 19. In addition, the tablet of Formulation 9 demonstrated acceptable swelling and firmness as shown in Table 20.

TABLE 16
Carbomer Type C Formulations

| | | | Amount (mg) | | | |
|---|---|---|---|---|---|---|
| | | | Formu- | Formu- | Formu- | Formu- |
| | Component | Function | lation 9 | lation 10 | lation 11 | lation 12 |
| Granular | Acamprosate | Active | 800 | 800 | 800 | 800 |
| Materials | Calcium | ingredient | | | | |
| | Avicel ® Ph 102 | Diluent | 100 | 100 | 100 | 100 |
| | Povidone K90 | Binder | 50 | 50 | 50 | 50 |
| | Colloidal Silicon | Glidant | 10 | 10 | 10 | 10 |
| | Dioxide | | | | | |
| Extra- | Carbopol ® 980P | Polymer | 60 | 90 | 30 | 140 |
| Granular | CMC 7 HF | Polymer | 40 | 40 | 40 | — |
| Materials | Starcap 1500 ® | Disintegrant | 40 | 10 | 70 | — |
| | Talc | Filler | 10 | 10 | 10 | 10 |
| | Magnesium | Lubricant | 10 | 10 | 10 | 10 |
| | Stearate | | | | | |

| | | | | |
|---|---|---|---|---|
| Tablet Shape | Circular | Circular | Circular | Circular |
| Tablet prior to coating | 1120 | 1120 | 1120 | 1120 |

TABLE 17
Release Profiles of Tablets at pH 1.0

| | Formu- | Formu- | Formu- | Formu- |
|---|---|---|---|---|
| | lation | lation | lation | lation |
| Release Profile | 9 | 10 | 11 | 12 |
| % Erosion (t = 2 h) | 31.7 | — | — | — |
| % Erosion (t = 6 h) | 48.6 | — | — | — |
| % Dissolution (t = 1 h) | 31.47 | 30.84 | 35.62 | 24.69 |
| % Dissolution (t = 2 h) | 45.51 | 42.08 | 49.91 | 33.99 |
| % Dissolution (t = 4 h) | 62.64 | 58.28 | 68.28 | 45.41 |
| % Dissolution (t = 6 h) | 75.03 | 72.24 | 80.64 | 55.22 |
| % Dissolution (t = 10 h) | 90.22 | 88.31 | 93.03 | 69.85 |
| % Dissolution (t = 12 h) | 95.15 | 93.24 | 97.32 | 77.93 |
| f2 | 73.34 | 92.2 | 56.06 | 44.1 |
| Y-intercept, SQRT | 3.8629 | 2.7275 | 6.5376 | 1.5118 |
| Slope, SQRT | 27.648 | 27.123 | 28.097 | 21.98 |
| R\hat{ }2, SQRT | 0.9904 | 0.9951 | 0.9774 | 0.9984 |

TABLE 18
Release Profiles of Tablets at pH 4.5

| Release Profile | Formulation 9 |
|---|---|
| % Erosion (t = 2 h) | 37.9 |
| % Erosion (t = 6 h) | 61.5 |
| % Dissolution (t = 1 h) | 36.58 |
| % Dissolution (t = 2 h) | 50.13 |
| % Dissolution (t = 4 h) | 66.59 |
| % Dissolution (t = 6 h) | 77.53 |
| % Dissolution (t = 10 h) | 95.12 |
| % Dissolution (t = 12 h) | 98.92 |
| f2 | 63.26 |
| Y-intercept, SQRT | 6.0574 |
| Slope, SQRT | 28.352 |
| R\hat{ }2, SQRT | 0.9857 |

TABLE 19
Release Profiles of Tablets at pH 6.8

| Release Profile | Formulation 9 |
|---|---|
| % Erosion (t = 2 h) | 38.28 |
| % Erosion (t = 6 h) | 58.45 |
| % Dissolution (t = 1 h) | 26.92 |
| % Dissolution (t = 2 h) | 31.17 |
| % Dissolution (t = 4 h) | 36.78 |
| % Dissolution (t = 6 h) | 42.01 |
| % Dissolution (t = 10 h) | 50.6 |
| % Dissolution (t = 12 h) | 53.87 |
| f2 | 58.6 |
| Y-intercept, SQRT | 6.8707 |
| Slope, SQRT | 14.326 |
| R\hat{ }2, SQRT | 0.9451 |

TABLE 20
% Swelling and Firmness of Tablets

| | Conditions | Formulation 9 |
|---|---|---|
| % Swelling | 0.1N HCl, pH 1.0 | 129% |
| (length) | Acetate Buffer, pH 4.5 | 117% |
| | Phosphate Buffer, pH 6.8 | 114% |
| % Swelling | 0.1N HCl, pH 1.0 | 172% |
| (thickness) | Acetate Buffer, pH 4.5 | 146% |
| | Phosphate Buffer, pH 6.8 | 148% |
| Firmness | 0.1N HCl, pH 1.0 | 4.5 |
| | Acetate Buffer, pH 4.5 | 3.8 |
| | Phosphate Buffer, pH 6.8 | 4.0 |

### Example 8: Formulations with Polyethylene Oxide

This Example summarizes the failure of compositions utilizing polyethylene oxide to meet certain performance requirements as described herein.

In particular, Table 21 presents the ingredients included in various provided formulations, prepared according to a two-step procedure as described above in Example 3.

TABLE 21

| | | | Amount (mg) | |
|---|---|---|---|---|
| | | | Formu- | Formu- |
| | Component | Function | lation 13 | lation 14 |
| Granular | Acamprosate | Active | 800 | 800 |
| Materials | Calcium | | | |
| | Avicel ® Ph 102 | Diluent | 100 | 100 |
| | Povidone K90 | Binder | 50 | 50 |
| | Colloidal Silicon | Glidant | 10 | 10 |
| | Dioxide | | | |
| Extra- | CMC 7 HF | Polymer | 40 | |
| Granular | Starcap 1500 | Disintegrant | 40 | |
| Materials | PEO 303 | Polymer | 60 | 140 |
| | Talc | Filler | 10 | 10 |
| | Magnesium | Lubricant | 10 | 10 |
| | Stearate | | | |

| | | |
|---|---|---|
| Tablet Shape | oval | oval |
| Tablet prior to coating | 1120 | 1120 |

Formulations 13 and 14 failed to meet the performance requirements at pH 1.0. Formulation 13 rapidly eroded in 0.1 N HCl, and % Erosion at t=2 h could not be measured. The dissolution profile of Formulation 13 also could not be measured over 12 hours, as only about 10% of the solid remained after 2 hours. Similarly, the dissolution profile of Formulation 14 could not be measured either, and after only 1 hour in 0.1 N HCl, the tablet fell apart when picked up. As a result, Formulations 13 and 14 were not further tested. These results demonstrate that not all polymers provide suitable formulations for the production of extended release tablets.

### Example 9: Formulations with Methylcellulose

This Example summarizes the failure of compositions utilizing methylcellulose to meet certain performance requirements as described herein.

In particular, Table 22 presents the ingredients included in various formulations, prepared according to a two-step procedure as described above in Example 3. As can be seen in the release profile data in Table 23, the 800 mg tablets from Formulation 15 performed similarly to the reference Formulation 2 with a dissolution f2 of 64.6. However, the tablet's gel was much weaker than the reference tablet and therefore Formulation 15 failed to meet the performance requirements. As a result, the 800 mg tablets of Formulation 15 were not further tested. The result below demonstrates that although a polymer may provide release data equivalent to the reference tablet, it may fail in other aspects of the requirements such as gel formation and therefore be unsuitable for the production of extended release tablets.

TABLE 22

| | | | Amount (mg) |
|---|---|---|---|
| | Component | Function | Formulation 15 |
| Granular | Acamprosate Calcium | Active | 800 |
| Materials | Avicel ® Ph 102 | Diluent | 100 |
| | Povidone K90 | Binder | 50 |
| | Colloidal Silicon | Glidant | 10 |
| | Dioxide | | |
| Extra- | METHOCEL ® K100M | Polymer | 140 |
| Granular | Talc | Filler | 10 |
| Materials | Magnesium Stearate | Lubricant | 10 |

| | |
|---|---|
| Tablet Shape | oval |
| Tablet prior to coating | 1120 |

TABLE 23
Release Profile of Tablet at pH 1.0

| Release Profile | Formulation 15 |
|---|---|
| % Erosion (t = 2 h) | 35.88 |
| % Erosion (t = 6 h) | (weak gel) |
| % Dissolution (t = h) | 35.08 |
| % Dissolution (t = 2 h) | 48.82 |
| % Dissolution (t = 4 h) | 65.24 |
| % Dissolution (t = 6 h) | 76.77 |
| % Dissolution (t = 10 h) | 89.44 |
| % Dissolution (t = 12 h) | 92.49 |
| f2 | 64.56 |
| Y-intercept, SQRT | 6.9063 |
| Slope, SQRT | 26.649 |
| R\hat{ }2, SQRT | 0.9752 |

### Example 10: Additional Formulations

This Example summarizes results obtained with compositions prepared with various polymer components (e.g., in a polymer matrix); as can be seen, several such compositions failed one or more performance parameters. For example, several compositions could not be effectively formulated into tablets with appropriate properties. For example, several compositions could not be effectively formulated into tablets that showed a release profile comparable to that of Formulation 2. Alternatively or additionally, several compositions could not be effectively formulated into tablets with appropriate erosion characteristics (e.g., prepared tablets showed significant erosion within 2 hours when placed under acidic conditions in 0.1 N HCl).

For example, certain compositions utilizing carbomer homopolymer type B and Poly(methyl acrylate-co-methyl methacrylate-co-methacrylic acid) 7:3:1, (i.e., Eudragit® FS 30 D) failed to meet certain performance requirements as described herein. Multiple different formulations were tested with various combinations of carbomer homopolymer type B and Poly(methyl acrylate-co-methyl methacrylate-co-methacrylic acid) 7:3:1, (i.e., Eudragit® FS 30 D). It was found that most such combinations failed due to unacceptable swelling and erosion results of the resulting tablets. Further, it was found that the above tested formulations failed to provide suitable dissolution profiles. It is worth noting that carbomer homopolymer type B was always present in an amount that was less than about 5.4 wt % of the tablet (and in a ratio of less than about 0.075:1 with respect to acamprosate calcium) in these failing tablets.

For example, certain compositions utilizing poly(ethylene) oxide (i.e., Polyox™ WSR-303) or cellulose ethers (i.e., METHOCEL™ K15M or METHOCEL™ K100M) failed to meet certain performance requirements as described herein. Multiple different formulations were tested with poly(ethylene) oxide (i.e., PEO 303 or Polyox™ WSR-303) or cellulose ethers (i.e., METHOCEL™ K15M or METHOCEL™ K100M). It was found that most of the formulations failed due to unacceptable swelling and erosion results of the resulting tablets. Further, it was found that the above tested formulations failed to provide suitable dissolution profiles. It is worth noting that the polyethylene oxide (i.e., PEO 303 or Polyox™ WSR-303) or cellulose ethers (i.e., METHOCEL™ K15M or METHOCEL™ K100M) was always present in an amount that was less than about 12.5 weight percent of the tablet (and in a ratio of less than 0.175:1 with respect to acamprosate) in these failing tablets.

In general, tablet compositions were considered to have failing swelling and erosion characteristics if the tablet did not remain intact over 12 hours in 0.1 N HCl (pH 1.0), acetate buffer (pH 4.5), and phosphate buffer (pH 6.8).

In general, compositions were considered to have failing release profile characteristics if they failed to provide a release profile comparable to 800 mg tablets of Example 1 (Formulation 2).

### Example 11: Preparation of Tablet Compositions Comprising Mini-Tablets

The process of Example 3 will be used to prepare mini-tablets comprising formulations described herein:
1. Wet granulation step: Materials (“granular materials”) will be weighed, sieved, and blended. Then, granulation will be effected using purified water as the granulating agent to form wet granules. The wet granules will be sieved and dried until the % LOD (loss on drying) is no more than 2%, resulting in the dry granules.
2. Extra-granulation step: The dry granules will be sieved and blended with the additional materials (“extra-granular materials”). The final blend of the granules and extra-granular materials will be compressed into mini-tablets.

The compressed mini-tablets will be evaluated for various parameters, including target weight, hardness, thickness, and friability.

In some embodiments, the process will further comprise a coating step.

In some embodiments, the process will further comprise an encapsulation step to provide a capsule comprising a plurality of mini-tablets.

### EQUIVALENTS

The herein described subject matter sometimes illustrates different methods, compositions and/or components contained within, or combined with, different other methods, compositions and/or components. It is to be understood that the various described methods, compositions, components, and combinations of the same are merely provided as non-limiting examples, and that in fact many others can be implemented which achieve the same purposes and/or functionality. Additional non-limiting examples of polymers that can be utilized with the formulations and compositions described herein are found in PHARMACEUTICAL POLYMERS in MARTIN'S PHYSICAL PHARMACY AND PHARMACEUTICAL SCIENCES, Sixth Edition 2010, by Patrick J. Sinko and published by Wolters Kluwer, ISBN: 9780781797665, at Chapter 20, pp 492-515; which is incorporated herein by reference in its entirety.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the embodiments of the technology.

The publications discussed herein are provided solely for their disclosure prior to the filing date of the present application. Nothing herein is to be construed as an admission that the present embodiments are not entitled to antedate such publication by virtue of prior invention. Further, the dates of publication provided may be different from the actual publication dates, which may need to be independently confirmed. The subject matter disclosed in the publications, including any methods, compositions, excipients (including ranges and dosages of the same), etc., are incorporated herein by reference in their entireties.

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. In a method of manufacturing an oval uncoated tablet composition comprising acamprosate in a pharmaceutically acceptable salt form and a carbomer homopolymer, wherein the acamprosate is present in a dose of at least 400 mg and the composition is prepared by blending:
(1) a preparation of granules with
(2) extra-granular materials,
the improvement that comprises including a cellulose derivative in each of (1) the preparation of granules, and (2) the extra-granular materials, so that the composition comprises:
the acamprosate in a dose of about 66 wt % to about 72 wt %;
the carbomer homopolymer in an amount of about 2 wt % to about 15 wt %; and
the cellulose derivative in an amount of about 5 wt % to about 9 wt %.

2. The improvement of claim 1, wherein the tablet composition is characterized in that, when it is placed in either acetate solution (pH 4.5) or HCl solution (pH 1.0) in vitro, it releases the acamprosate at a rate that is approximately linear with the square root of time, and with comparable rate constants in each solution.

3. The improvement of claim 1 or 2, wherein the tablet composition is characterized by a % swelling in the length of the tablet composition in a range of about 100% to about 180% when placed in 0.1 N HCl (pH 1.0), acetate solution (pH 4.5) or phosphate solution (pH 6.8).

4. The improvement of any one of claims 1-3, wherein the tablet composition is characterized by a % swelling in the thickness of the tablet composition in a range of about 100% to about 175% when placed in 0.1 N HCl (pH 1.0), acetate solution (pH 4.5) or phosphate solution (pH 6.8).

5. The improvement of any one of claims 1-4, wherein the total weight of the tablet composition is about 1000 mg to about 1280 mg.

6. The improvement of any one of claims 1-5, wherein the total weight of the tablet composition is about 1120 mg to about 1200 mg.

7. The improvement of any one of claims 1-6, wherein the total weight of the tablet composition is about 1140 mg to about 1180 mg.

8. The improvement of any one of claims 1-7, wherein the dose of the acamprosate is about 660 mg to about 921 mg.

9. The improvement of any one of claims 1-8, wherein the dose of the acamprosate is about 740 mg to about 864 mg.

10. The improvement of any one of claims 1-9, wherein the dose of the acamprosate is about 752 mg to about 849 mg.

11. The improvement of any one of claims 1-10, wherein the dose of the acamprosate is about 800 mg.

12. The improvement of any one of claims 1-11, wherein the carbomer homopolymer is carbomer homopolymer type B.

13. The improvement of any one of claims 1-12, wherein the carbomer homopolymer type B is in an amount of about 3 wt % to about 7 wt %.

14. The improvement of any one of claims 1-13, wherein the carbomer homopolymer type B is in an amount of about 60 mg.

15. The improvement of any one of claims 1-14, wherein the cellulose derivate is carboxymethyl cellulose (CMC 7HF).

16. The improvement of any one of claims 1-15, wherein the cellulose derivative is in an amount of about 6 wt % to about 8 wt %.

17. The improvement of any one of claims 1-16, wherein the cellulose derivative is in an amount of about 60 mg to about 100 mg.

18. The improvement of any one of claims 1-17, wherein the cellulose derivative is in an amount of about 80 mg.

19. The improvement of any one of claims 1-18, wherein the tablet composition comprises any one of Formulations 5-12 from Tables 10, 11, and 16.

20. The improvement of any one of claims 1-19, wherein the tablet composition comprises Formulation 5 from Table 10.

21. An uncoated tablet composition comprising:
acamprosate in a pharmaceutically acceptable salt form in a dose of about 66 wt % to about 72 wt %;
carbomer homopolymer type B in an amount of about 3 wt % to about 7 wt %;
a cellulose derivative in an amount of about 5 wt % to about 9 wt %; and
optionally one or more pharmaceutically acceptable excipients.

22. The tablet composition of claim 21, wherein the tablet composition is oval.

23. The tablet composition of claim 21 or 22, wherein the tablet composition is characterized in that, when it is placed in either acetate solution (pH 4.5) or HCl solution (pH 1.0) in vitro, it releases the acamprosate at a rate that is approximately linear with the square root of time, and remains comparable.

24. The tablet composition of any one of claims 21-23, wherein the tablet composition is characterized by a % swelling in the length of the tablet composition in a range of about 100% to about 130% when placed in 0.1 N HCl (pH 1.0), acetate solution (pH 4.5) or phosphate solution (pH 6.8).

25. The tablet composition of any one of claims 21-24, wherein the tablet composition is characterized by a % swelling in the thickness of the tablet composition in a range of about 100% to about 180% when placed in 0.1 N HCl (pH 1.0), acetate solution (pH 4.5) or phosphate solution (pH 6.8).

26. The tablet composition of any one of claims 21-25, wherein the total weight of the tablet composition is about 1000 mg to about 1280 mg.

27. The tablet composition of any one of claims 21-26, wherein the total weight of the tablet composition is about 1120 mg to about 1200 mg.

28. The tablet composition of any one of claims 21-27, wherein the total weight of the tablet composition is about 1140 mg to about 1180 mg.

29. The tablet composition of any one of claims 21-28, wherein the dose of the acamprosate is about 660 mg to about 921 mg.

30. The tablet composition of any one of claims 21-29, wherein the dose of the acamprosate is about 740 mg to about 864 mg.

31. The tablet composition of any one of claims 21-30, wherein the dose of the acamprosate is about 752 mg to about 849 mg.

32. The tablet composition of any one of claims 21-31, wherein the dose of the acamprosate is about 800 mg.

33. The tablet composition of any one of claims 21-32, wherein the carbomer homopolymer type B is in an amount of about 50 mg to about 70 mg.

34. The tablet composition of any one of claims 21-33, wherein the carbomer homopolymer type B is in an amount of about 60 mg.

35. The tablet composition of any one of claims 21-34, wherein the cellulose derivate is carboxymethyl cellulose (CMC 7HF).

36. The tablet composition of any one of claims 21-35, wherein the cellulose derivative is in an amount of about 6 wt % to about 8 wt %.

37. The tablet composition of any one of claims 21-36, wherein the cellulose derivative is in an amount of about 60 mg to about 100 mg.

38. The tablet composition of any one of claims 21-37, wherein the cellulose derivative is in an amount of about 80 mg.

39. The tablet composition of any one of claims 21-38, wherein the tablet composition comprises Formulation 5 from Table 10.

40. An uncoated tablet composition comprising:
acamprosate in a pharmaceutically acceptable salt form, in a dose of about 66 wt % to about 72 wt %;
carbomer homopolymer type B in an amount of about 6 wt % to about 10 wt %;
gelatin type B in an amount of about 2 wt % to about 5 wt %; and
optionally one or more pharmaceutically acceptable excipients.

41. The tablet composition of claim 40, wherein the tablet composition is oval.

42. The tablet composition of claim 40 or 41, wherein the tablet composition is characterized in that, when it is placed in either acetate solution (pH 4.5) or HCl solution (pH 1.0) in vitro, it releases the acamprosate at a rate that is approximately linear with the square root of time, and remains comparable.

43. The tablet composition of any one of claims 40-42, wherein the tablet composition is characterized by a % swelling in the length of the tablet composition in a range of about 100% to about 130% when placed in 0.1 N HCl (pH 1.0), acetate solution (pH 4.5), or phosphate solution (pH 6.8).

44. The tablet composition of any one of claims 40-43, wherein the tablet composition is characterized by a % swelling in the thickness of the tablet composition in a range of about 100% to about 180% when placed in 0.1 N HCl (pH 1.0), acetate solution (pH 4.5), or phosphate solution (pH 6.8).

45. The tablet composition of any one of claims 40-44, wherein the total weight of the tablet composition is about 1000 mg to about 1280 mg.

46. The tablet composition of any one of claims 40-45, wherein the total weight of the tablet composition is about 1080 mg to about 1160 mg.

47. The tablet composition of any one of claims 40-46, wherein the total weight of the tablet composition is about 1100 mg to about 1140 mg.

48. The tablet composition of any one of claims 40-47, wherein the dose of the acamprosate is about 660 mg to about 921 mg.

49. The tablet composition of any one of claims 40-48, wherein the dose of the acamprosate is about 713 mg to about 835 mg.

50. The tablet composition of any one of claims 40-49, wherein the dose of the acamprosate is about 726 mg to about 821 mg.

51. The tablet composition of any one of claims 40-50, wherein the dose of the acamprosate is about 800 mg.

52. The tablet composition of any one of claims 40-51, wherein the gelatin type B is in an amount of about 3 wt % to about 4 wt %.

53. The tablet composition of any one of claims 40-52, wherein the gelatin type B is in an amount of about 40 mg.

54. The tablet composition of any one of claims 40-53, wherein the tablet composition comprises Formulation 6 from Table 11.

55. The tablet composition of any one of claims 40-53, wherein the tablet composition comprises Formulation 7 from Table 11.

56. The tablet composition of any one of claims 40-53, wherein the tablet composition comprises Formulation 8 from Table 11.

57. An uncoated tablet composition comprising:
acamprosate in a pharmaceutically acceptable salt form, in a dose of about 66 wt % to about 72 wt %;
carbomer homopolymer type C, in an amount of about 2 wt % to about 15 wt %; and
optionally one or more pharmaceutically acceptable excipients.

58. The tablet composition of claim 57, wherein the tablet composition is oval.

59. The tablet composition of claim 57 or 58, wherein the tablet composition is characterized in that, when it is placed in either acetate solution (pH 4.5) or HCl solution (pH 1.0) in vitro, it releases the acamprosate at a rate that is approximately linear with the square root of time, and remains comparable.

60. The tablet composition of any one of claims 57-59, wherein the tablet composition is characterized by a % swelling in the length of the tablet composition in a range of about 100% to about 130% when placed in 0.1 N HCl (pH 1.0), acetate solution (pH 4.5) or phosphate solution (pH 6.8).

61. The tablet composition of any one of claims 57-60, wherein the tablet composition is characterized by a % swelling in the thickness of the tablet composition in a range of about 100% to about 180% when placed in 0.1 N HCl (pH 1.0), acetate solution (pH 4.5) or phosphate solution (pH 6.8).

62. The tablet composition of any one of claims 57-61, wherein the total weight of the tablet composition is about 1000 mg to about 1280 mg.

63. The tablet composition of any one of claims 57-62, wherein the total weight of the tablet composition is about 1080 mg to about 1160 mg.

64. The tablet composition of any one of claims 57-63, wherein the total weight of the tablet composition is about 1100 mg to about 1140 mg.

65. The tablet composition of any one of claims 57-64, wherein the dose of the acamprosate is about 660 mg to about 921 mg.

66. The tablet composition of any one of claims 57-65, wherein the dose of the acamprosate is about 713 mg to about 835 mg.

67. The tablet composition of any one of claims 57-66, wherein the dose of the acamprosate is about 726 mg to about 821 mg.

68. The tablet composition of any one of claims 57-67, wherein the dose of the acamprosate is about 800 mg.

69. The tablet composition of any one of claims 57-68, wherein the carbomer homopolymer type C is in an amount of about 4 wt % to about 12 wt %.

70. The tablet composition of any one of claims 57-69, wherein the carbomer homopolymer type C is in an amount of about 30 mg to about 140 mg.

71. The tablet composition of any one of claims 57-70, wherein the carbomer homopolymer type C is in an amount of about 60 mg.

72. The tablet composition of any one of claims 57-71, wherein the carbomer homopolymer type C is in an amount of about 90 mg.

73. The tablet composition of any one of claims 57-72, wherein the tablet composition comprises Formulation 9 from Table 16.

74. The tablet composition of any one of claims 57-72, wherein the tablet composition comprises Formulation 10 from Table 16.

75. The tablet composition of any one of claims 57-72, wherein the tablet composition comprises Formulation 11 from Table 16.

76. The tablet composition of any one of claims 57-72, wherein the tablet composition comprises Formulation 12 from Table 16.

77. An oral dosage form comprising one or more tablet compositions of any one of claims 21-76.

78. The oral dosage form of claim 77, comprising one tablet composition.

79. The oral dosage form of claim 78, wherein the one tablet composition is less than or equal to 22 mm in the largest dimension.

80. The oral dosage form of claim 78 or 79, wherein the one tablet composition is less than or equal to 20 mm in the largest dimension.

81. The oral dosage form of any one of claims 78-80, wherein the one tablet composition is less than or equal to 13 mm in the largest dimension.

82. The oral dosage form of any one of claims 78-81, wherein the one tablet composition is less than or equal to 8 mm in the largest dimension.

83. The oral dosage form of claim 77, comprising a plurality of tablet compositions.

84. The oral dosage form of claim 83, wherein each tablet composition is less than or equal to 6 mm in the largest dimension.

85. The oral dosage form of claim 83 or 84, wherein each tablet composition is less than or equal to 4 mm in the largest dimension.

86. The oral dosage form of any one of claims 83-85, wherein each tablet composition is less than or equal to 2 mm in the largest dimension.

87. The oral dosage form of any one of claims 83-86, wherein each tablet composition is less than or equal to 1 mm in the largest dimension.

88. The oral dosage form of any one of claims 83-87, wherein the dose of acamprosate is at least 400 mg.

89. The oral dosage form of any one of claims 83-88, wherein the dose of acamprosate is at least 800 mg.

90. The oral dosage form of any one of claims 83-89, wherein the dose of acamprosate is about 800 mg.

91. The oral dosage form of any one of claims 83-90, wherein the dose of acamprosate is about 1000 mg.

92. A method of preparing an uncoated tablet composition comprising acamprosate in a pharmaceutically acceptable salt form, comprising:
(i) blending a preparation of granules that comprises the acamprosate with extra-granular materials that comprise a carbomer homopolymer to form a blended material, wherein each of the granules and the extra-granular materials comprises a cellulose derivative material; and
(ii) compressing the blended material to form the uncoated tablet composition.

93. The method of claim 92, wherein the method further comprises a step of first providing the preparation of granules.

94. The method of claim 92 or 93, wherein the step of providing the preparation of granules comprises (a) forming wet granules and (b) drying the wet granules to form the preparation of granules.

95. The method of any one of claims 92-94, wherein the method further comprises a step of coating the uncoated tablet composition.

96. The method of any one of claims 92-95, wherein the preparation of granules comprises about 2 wt % to about 6 wt % of carboxymethyl cellulose

97. The method of any one of claims 92-96, wherein the preparation of granules comprises about 2 wt % to about 6 wt % of carboxymethyl cellulose and the extragranular materials comprise about 20 wt % to about 30 wt % of carboxymethyl cellulose, so that the tablet composition comprises a total of about 6 wt % to about 8 wt % of carboxymethyl cellulose.

98. The method of any one of claims 92-97, wherein the preparation of granules comprises the acamprosate in an amount of about 70 wt % to about 90 wt %.

99. The method of any one of claims 92-98, wherein the preparation of granules comprises the acamprosate in an amount of about 80 wt %.

100. The method of any one of claims 92-99, wherein the extra-granular materials comprise a carbomer homopolymer in an amount of about 15 wt % to about 90 wt %.

101. The method of any one of claims 92-100, wherein the extra-granular materials comprise carbomer homopolymer type B in an amount of about 35 wt % to about 60 wt %.

102. The method of any one of claims 92-101, wherein the extra-granular materials comprise carbomer homopolymer type B in an amount of about 37.5 wt %.

103. The method of any one of claims 92-101, wherein the extra-granular materials comprise carbomer homopolymer type C in an amount of about 15 wt % to about 90 wt %.

104. The method of any one of claims 92-103, wherein the tablet composition comprises any one of Formulations 5-12 from Tables 9, 10, and 15.

105. The method of any one of claims 92-104, wherein the wet granules are dried until % loss on drying (LOD) is no more than 2%.

106. The method of any one of claims 92-105, wherein the tablet composition is oval.

107. The method of any one of claims 92-106, wherein the tablet composition is characterized in that, when it is placed in either acetate solution (pH 4.5) or HCl solution (pH 1.0) in vitro, it releases the acamprosate at a rate that is approximately linear with the square root of time, and remains comparable.

108. The method of any one of claims 92-107, wherein the tablet composition has a release profile that is comparable to that of Formulation 2.

109. The method of any one of claims 92-108, wherein the tablet composition is characterized by a % swelling in the length of the tablet composition in a range of about 100% to about 130% when placed in 0.1 N HCl (pH 1.0), acetate solution (pH 4.5) or phosphate solution (pH 6.8).

110. The method of any one of claims 92-109, wherein the tablet composition is characterized by a % swelling in the thickness of the tablet composition in a range of about 100% to about 180% when placed in 0.1 N HCl (pH 1.0), acetate solution (pH 4.5) or phosphate solution (pH 6.8).

111. An uncoated tablet composition, wherein the tablet composition is obtained by a method of any one of claims 92-110.

112. An oral dosage form comprising one or more tablet compositions obtained by a method of any one of claims 92-110.

113. A method of administering a therapeutically effective dose of acamprosate to a patient, the method comprising a step of:
administering to the patient a tablet composition of any one of claim 21-76 or 111 or an oral dosage form of any one of claim 77-91 or 112.

114. The method of claim 113, wherein the step of administering comprises administering 1 to 4 tablet compositions or oral dosage forms per day.

115. The method of claim 113 or 114, wherein the step of administering comprises administering 1 to 4 tablet compositions or oral dosage forms once per day.

116. The method of any one of claims 113-115, wherein the step of administering comprises administering 1 or 2 tablet compositions or oral dosage forms once per day.

117. The method of claim 113 or 114, wherein the step of administering comprises administering 1 or 2 tablet compositions twice per day.

118. The method of any one of claims 113-117, wherein the patient is in the fed mode.

119. The method of any one of claims 113-117, wherein the patient is in the fasted mode.

120. The method of any one of claims 113-119, wherein the step of administering comprises administering to a patient with a neuropsychiatric disorder selected from the group consisting of psychogenic nonepileptic seizures (PNES), tardive dyskinesia (TD), tardive akathisia, dystonia, blepharospasm, levodopa-induced dyskinesia (LID) in patients with Parkinson's disease, dyskinetic movements in Rett's Syndrome, dyskinetic movements in DiGeorge Syndrome, dyskinetic movements and dystonia in Wilson's disease, post-hypoxic myoclonus, simple tics, Tourette Syndrome (TS), obsessive-compulsive disorder (OCD), posttraumatic stress disorder (PTSD), symptoms of schizophrenia, depression, bipolar disorder, autism spectrum disorders, autistic symptoms in Fragile X syndrome, alcoholism, tinnitus, generalized anxiety disorder, and repetitive and stereotypic self-injurious behaviors (SIB) in persons with developmental disabilities such as biting, skin-picking, hitting oneself, and head-banging.

121. The method of any one of claims 113-120, wherein the tablet composition or oral dosage form comprises any one of Formulations 5-12 from Tables 10, 11, and 16.

122. The method of any one of claims 113-121, wherein the tablet composition or oral dosage form comprises Formulation 5 from Table 10.
